SEQUENCE LISTING (17 sequences)

SEQ ID NO: 1 - DNA, 399 bp, Artificial Sequence
  LPA-KIV9 Heavy chain
  CDS (1)..(399)
atggaatggagctgggtctttctcttcctcctgtcagtaactgcaggtgtccaatcccaggttcaattgcagcagtctggggctgagctggtgaggcctggggcttcagtgaaactgtcctgcaaggctttgggctacacatttactgactatgaattgcactggctgaagcagacacctgtgcatggcctggaatggattggagctgttcatccaggaagcggtggttctgcctacaatcagaacttcaaggacaaggccacactgactgcagacaaatcctccagcacagcctatatggagctcagcagcctgacatctgaggactctgctgtctattactgtacaagagaaggacctttctactggggccaaggcaccactctcacagtctcctca

SEQ ID NO: 2 - protein, 133 aa, Artificial Sequence
  Synthetic Construct
MEWSWVFLFLLSVTAGVQSQVQLQQSGAELVRPGASVKLSCKALGYTFTDYELHWLKQTPVHGLEWIGAVHPGSGGSAYNQNFKDKATLTADKSSSTAYMELSSLTSEDSAVYYCTREGPFYWGQGTTLTVSS

SEQ ID NO: 3 - DNA, 24 bp, Artificial Sequence
  LPA-KIV9 Heavy chain CDR1 coding sequence
  CDS (1)..(24)
ggctacacatttactgactatgaa

SEQ ID NO: 4 - protein, 8 aa, Artificial Sequence
  Synthetic Construct
GYTFTDYE

SEQ ID NO: 5 - DNA, 24 bp, Artificial Sequence
  LPA-KIV9 Heavy chain CDR2 coding sequence
  CDS (1)..(24)
gttcatccaggaagcggtggttct

SEQ ID NO: 6 - protein, 8 aa, Artificial Sequence
  Synthetic Construct
VHPGSGGS

SEQ ID NO: 7 - DNA, 27 bp, Artificial Sequence
  LPA-KIV9 Heavy chain CDR3 coding sequence
  CDS (1)..(27)
tgtacaagagaaggacctttctactgg

SEQ ID NO: 8 - protein, 9 aa, Artificial Sequence
  Synthetic Construct
CTREGPFYW

SEQ ID NO: 9 - DNA, 393 bp, Artificial Sequence
  LPA-KIV9 Light chain
  CDS (1)..(393)
atgaagttgcctgttaggctgttggtgctgatgttctggattcctgcttccagtagtgatgttgtgatgacccaaactccactctccctgcctgtcagtcttggagatcaagcctccatctcttgtagatctagtcagagccttgtacacagtaatggaaacgcctatttacattggtacctgcagaagccaggccagtctccaaagctcctgatctacaaagtttccaaccgattttctggggtcccagacaggttcagtggcagtggatcagggacaaatttcacactcaagatcagtagagtggaggctgaggatctgggaatttatttctgctctcaaagtacacatgttccgctcacgttcggtgctgggaccaagctggagctgaaa

SEQ ID NO: 10 - protein, 131 aa, Artificial Sequence
  Synthetic Construct
MKLPVRLLVLMFWIPASSSDVVMTQTPLSLPVSLGDQASISCRSSQSLVHSNGNAYLHWYLQKPGQSPKLLIYKVSNRFSGVPDRFSGSGSGTNFTLKISRVEAEDLGIYFCSQSTHVPLTFGAGTKLELK

SEQ ID NO: 11 - DNA, 33 bp, Artificial Sequence
  LPA-KIV9 Light chain CDR1 coding sequence
  CDS (1)..(33)
cagagccttgtacacagtaatggaaacgcctat

SEQ ID NO: 12 - protein, 11 aa, Artificial Sequence
  Synthetic Construct
QSLVHSNGNAY

SEQ ID NO: 13 - DNA, 9 bp, Artificial Sequence
  LPA-KIV9 Light chain CDR2 coding sequence
  CDS (1)..(9)
aaagtttcc

SEQ ID NO: 14 - protein, 3 aa, Artificial Sequence
  Synthetic Construct
KVS

SEQ ID NO: 15 - DNA, 33 bp, Artificial Sequence
  LPA-KIV9 Light chain CDR3 coding sequence
  CDS (1)..(33)
tgctctcaaagtacacatgttccgctcacgttc

SEQ ID NO: 16 - protein, 11 aa, Artificial Sequence
  Synthetic Construct
CSQSTHVPLTF

SEQ ID NO: 17 - protein, 23 aa, Artificial Sequence
  Lp(a) antigenic peptide
CSETESGVLETPTVVPVPSMEAH

(19) United States
(12) Patent Application Publication
(10) Pub. No.: US 2025/0321239 A1
(43) Pub. Date: Oct. 16, 2025

(54) HIGH-THROUGHPUT METHOD FOR LP(A)-CHOLESTEROL QUANTITATION

(71) Applicant: The Regents of the University of California, Oakland, CA (US)

(72) Inventors: Sotirios Tsimikas, Rancho Santa Fe, CA (US); Calvin Yeang, La Jolla, CA (US)

(21) Appl. No.: 18/029,606

(22) PCT Filed: Oct. 5, 2021
(86) PCT No.: PCT/US2021/053633

§ 371 Date: Mar. 30, 2023

Related U.S. Application Data
(60) Provisional application No. 63/087,700, filed on Oct. 5, 2020.

(51) Int. Cl. G01N 33/92; G01N 21/78; G01N 33/543

(52) U.S. Cl. CPC G01N 33/92; G01N 21/78; G01N 33/54326

(57) ABSTRACT

The disclosure provides methods and composition for measuring LDL-Cholesterol (LDL-C) in a sample. The method includes removing Lp(a)-cholesterol in order to determine a more accurate value of circulating LDL-C.

### CROSS REFERENCE TO RELATED APPLICATIONS

This application claims priority to U.S. Provisional Application No. 63/087,700, filed Oct. 5, 2020; the disclosures of which are incorporated herein by reference.

### TECHNICAL FIELD

The disclosure provides methods and compositions to determine lipoprotein (a)-Cholesterol (Lp(a)-C) content, methods of diagnosing and treating diseases and disorders associated with Lp(a)-C.

### INCORPORATION BY REFERENCE OF SEQUENCE LISTING

Accompanying this filing is a Sequence Listing entitled “Sequence-Listing ST25.txt”, created on Oct. 5, 2021, and having 11,259 bytes of data, machine formatted on IBM-PC, MS-Windows operating system. The sequence listing is hereby incorporated herein by reference in its entirety for all purposes.

### BACKGROUND

Lipoprotein (a) [Lp(a)] is composed of apolipoprotein (a) covalently bound to apolipoprotein B-100. The apolipoprotein (a) protein displays wide size heterogeneity due to a variable number of kringle KIV2 repeats among individuals and populations.

Apolipoprotein (a) consists of 10 unique kringle IV repeats that are present in one copy, except KIV2 which is present in a variable number of identical copies (1 to >40). It also contains one copy of KV and an inactive protease-like domain. Plasma Lp(a) levels are genetically determined by the production rate of apolipoprotein (a) in hepatocytes, with isoforms containing a small number of KIV2 repeats being secreted more efficiently, leading to an inverse association of KIV2 repeat number and plasma Lp(a) levels.

Low density lipoprotein cholesterol (LDL-C) is routinely used to assess LDL mediated cardiovascular disease (CVD) risk and response to therapy. All “LDL-C” assays used in clinical practice, including the reference method “beta-quantification”, measure the cholesterol content of both LDL and lipoprotein (a) [Lp(a)-C], the latter which contains cholesterol in its LDL moiety. For patients with even modest elevations of Lp(a), Lp(a)-C can constitute a significant portion of measured “LDL-C.” Without this methodological confounder, the correct LDL-C in such patients can be significantly lower than the laboratory measurement of “LDL-C”.

### SUMMARY

Elevated Lipoprotein (a) (Lp(a)), and independent and genetically determined risk factor for cardiovascular disease including coronary artery disease, stroke, and aortic valve stenosis, is present in ˜2 billion people. It is underappreciated that LDL-C, as determined by all clinically available assays, is inaccurate due to contamination by the cholesterol content of Lp(a) (Lp(a)-C). There is a need for more precise, personalized Lp(a)-C and corrected LDL-C measurements for accurate CVD risk assessment and treatment. The disclosure provides methods and compositions that measure Lp(a)-C and non-Lp(a)-C in clinical blood samples using a sensitive and specific high throughput system that complements the existing lipid panel. By determining the Lp(a)-C using the methods and compositions of the disclosure one can then subtract this from “LDL-C” measured by a conventional lipid panel to obtain a corrected LDL-C. Both Lp(a)-C and corrected LDL-C can be used for cardiovascular risk assessment.

The disclosure provides a method of assaying lipoprotein (a)-cholesterol (Lp(a)-C) in a sample, the method comprising contacting a sample with a binding molecule that specifically binds to apolipoprotein (a) (apo (a)) to obtain an apo (a)-binding molecule complexes; isolating the apo (a)-binding molecule complexes; performing an enzymatic colorimetric assay on the isolated apo (a)-binding molecule complexes to measure cholesterol. In one embodiment, the method further comprises removing the apo (a)-binding molecule complexes and measuring the absorbance of the sample. In another embodiment, the binding molecule comprises light chain and heavy chain complement determining regions (CDRs) of an LPA4 antibody. In still another embodiment, the binding molecule is an antibody or antibody fragment. In a further embodiment, the antibody or antibody fragment recognizes and binds to lipoprotein (a), wherein the antibody or antibody fragment comprises a variable heavy chain (V_{H}) domain and/or a variable light chain (V_{L}) domain, and wherein (a) the V_{H} domain comprises an amino acid sequence that includes complementarity determining regions (CDRs) selected from the group consisting of: SEQ ID NO: 4 or variants thereof; SEQ ID NO: 6 or variants thereof; and SEQ ID NO: 8 or variants thereof; and (b) the V_{L} domain comprises an amino acid sequence that includes complementarity determining regions (CDRs) selected from the group consisting of: SEQ ID NO: 12 or variants thereof; SEQ ID NO: 14 or variants thereof; and SEQ ID NO: 16 or variants thereof. In still another or further embodiment, the V_{H} domain comprises an amino acid sequence of SEQ ID NO:2, and/or the V_{L} domain comprises an amino acid sequence of SEQ ID NO: 10. In still another or further embodiment, the antibody or antibody fragment is selected from the group consisting of an antibody or scFv with heavy and light chain domains comprising the complementarity determining regions of SEQ ID NO: 4, 6, 8, 12, 14, and 16. In still another embodiment, the antibody or antibody fragment binds to an epitope having the sequence of SEQ ID NO: 17. In another embodiment, the binding molecule is linked to a substrate. In a further embodiment, the substrate is an ELISA plate. In another embodiment, the substrate is a bead. In a further embodiment, the bead is a magnetic bead. In yet another embodiment, the sample is from a subject undergoing therapy for high cholesterol.

The disclosure also provides a method of assaying lipoprotein (a)-cholesterol (Lp(a)-C) in a sample, the method comprising contacting a sample with an antibody or antibody fragment that specifically binds to apolipoprotein (a) (Apo (a)) to obtain and Apo (a)-antibody complex; isolating the apo (a)-antibody complexes; and measuring the amount of cholesterol in the isolated Apo (a)-antibody complex. In another embodiment, the antibody is bound to a substrate. In a further embodiment, the method further comprises washing the substrate to remove non-bound materials. In yet another embodiment, the antibody is bound to a bead. In a further embodiment, the bead is magnetic. In still a further embodiment, the Apo (a)-antibody complex is isolated using a magnet. In still another embodiment, of any of the foregoing, the cholesterol is measured using a colorimetric enzymatic assay. In a further embodiment, the absorbance of the colorimetric assay is compared to a control curve. In another embodiment, the method determines the level of cholesterol in an Apo (a) containing fraction of plasma. In still another embodiment, the antibody or antibody fragment comprises a variable heavy chain (V_{H}) domain and/or a variable light chain (V_{L}) domain, and wherein (a) the V_{H} domain comprises an amino acid sequence that includes complementarity determining regions (CDRs) of SEQ ID NO: 4 or variants thereof; SEQ ID NO: 6 or variants thereof; and SEQ ID NO: 8 or variants thereof; and (b) the V_{L} domain comprises an amino acid sequence that includes complementarity determining regions (CDRs) of SEQ ID NO: 12 or variants thereof; SEQ ID NO: 14 or variants thereof; and SEQ ID NO: 16 or variants thereof. In yet another embodiment, the antibody or antibody fragment is selected from the group consisting of an antibody or scFv with heavy and light chain domains comprising the complementarity determining regions of SEQ ID NO:4, 6, 8, 12, 14, and 16.

The disclosure also provides a method of assaying low density lipoprotein cholesterol (LDL-C) in a sample, the method comprising contacting a plasma sample with a composition or article of manufacture comprising a binding agent linked to a carrier, wherein the binding agent specifically binds to Lp(a) and wherein the carrier separates bound Lp(a) (Lp(a)-C fraction) from a soluble fraction of the plasma and measuring the amount of cholesterol in the soluble fraction of the plasma thereby obtaining LDL-C value. In a further embodiment, the binding agent is an antibody that specifically binds to Lp(a). In another or further embodiment, the antibody is a polyclonal antibody. In still another or further embodiment, the antibody is a monoclonal antibody. In a further embodiment, the antibody is LPA4. In still another embodiment, the antibody comprises one or more CDRs having sequences of SEQ ID NO: 4, 6, 8, 12, 14, and/or 16. In yet another embodiment, the method further comprises measuring the amount of cholesterol in the Lp(a)-C fraction to obtain a Lp(a)-C value. In yet another embodiment, the method further comprises measuring the amount of total cholesterol in the sample or a corresponding sample prior to contacting the sample, with the composition or article of manufacture to obtain an LDL-C value. In a further embodiment, the method further comprises measuring the amount of total cholesterol in the sample or a corresponding sample prior to contacting the sample, with the composition or article of manufacture to obtain an LDL-C. In a further embodiment, the Lp(a)-C value is subtracted from the total cholesterol value to obtain a corrected LDL-C value. In still another embodiment, the composition comprises a bead linked to the binding agent. In a further embodiment, the bead is a magnetic bead. In another embodiment, the bead is a biotin or streptavidin bead. In still another embodiment, the article of manufacture is a substrate comprising the binding agent. In a further embodiment, the substrate is an ELISA substrate or a microwell plate.

The disclosure also provides a kit or an article of manufacture for carrying out the method of described herein compartmentalized to containing and Lp(a) binding agent, cholesterol colorimetric reagents and the like.

### DESCRIPTION OF DRAWINGS

FIG. 1 shows a schematic of the Lp(a)-C assay. Lp(a) in plasma is affinity captured by LPA4 covalently bound to magnetic dynabeads (LPA4-dynabeads) and separated from other cholesterol carrying lipoproteins (depicted as yellow circles in the left panel) in each well by magnetic extraction and washes. Then, an enzymatic, colorimetric cholesterol reagent is added to each well, generating a color (i.e., red) with intensity proportional to the amount of cholesterol present. Following a 5-minute incubation period to ensure all cholesterol on Lp(a) has been processed, LPA4-dynabeads are extracted by magnet, and the absorbance at 500 nm (primary) and 700 nm (background) quantified.

FIG. 2 shows efficacy of Lp(a) affinity capture using LPA4-dynabeads. The amount of Lp(a) remaining in each 15 ul aliquot of plasma following incubation with increasing amounts of LPA4-dynabeads. Lp(a) was quantified by ELISA and expressed as a percentage of Lp(a) in plasma not exposed to LPA4-dynabeads. Each data point represents the mean±S.D. of 3 independent experiments.

FIG. 3A-D shows Lp(a)-C assay sensitivity and linearity across a range of Lp(a) molar concentrations. (A) Lp(a)-C measured in plasma from mice that lack Lp(a) but has LDL-C, spiked-in with purified Lp(a), and (B) expressed as a percentage of total cholesterol directedly measured on purified Lp(a). The dotted lines in panel B delineate 120% and 80% recovery rates. Lp(a)-C measured in serial dilutions of plasma with Lp(a) particle number of 85.0 nM (C) and 355.0 nM (D). Each data point represents the mean±S.D. of 3 independent experiments.

FIG. 4A-F shows the relationship between Lp(a)-C and Lp(a) molar concentrations in all individuals (A), those treated with IONIS-APO (a) Rx ASO (B), and those treated with placebo ASO (C). Relationship between % Lp(a)-C and Lp(a) mass in all individuals (D), those treated with IONIS-APO (a) Rx ASO (E), and those treated with placebo ASO (F). Data from baseline, trough, and recovery timepoints are represented in each panel. In panels C and F, each color in the legend references one individual. Correlation by Spearman's rho (r).

FIG. 5 shows quantification of apolipoprotein (a) [apo (a)], apolipoprotein B-100 (apoB100), apolipoprotein AI (apoAI) by ELISA, and total protein in each fraction eluted from the SW-400 gel filtration column as part of the Lp(a) purification process. Relative light units (RLUs) associated with the apoAI ELISA are expressed on a secondary (right) axis due to much lower values compared to apo (a) and apoB100. The fractions delineated by black lines correspond to those pooled for pure Lp(a).

FIG. 6A-B shows the purity of Lp(a) preparation. Agarose gel electrophoresis and lipid staining of plasma, purified LDL, purified HDL and purified Lp(a) (A) from the same donor. SDS PAGE of purified Lp(a) and LDL, under reducing or nonreducing conditions (B). The prominent dark band immediately below the well likely represents aggregated apoB100. The expected molecular weight for apoB100 is 550 kDa and apo (a) is at least 325 kDa [major isoform of 24 kringles+protease domain+glycosylation]. The minor apo (a) isoform (separately assessed to be <5% of total apo (a)) is not appreciated with these loading conditions. BME=beta-mercaptoethanol.

FIG. 7 shows LPA4-dynabead immunoprecipitated from human plasma containing Lp(a) compared to mouse plasma containing human apoB100/LDL but not Lp(a). BME=beta-mercaptoethanol. Arrows denote expected molecular weights of the intact LPA4 IgG and its heavy chains when reduced.

FIG. 8A-B shows (A) Frequency distribution of Lp(a) molar concentration and (B) direct Lp(a)-C at the baseline timepoint in the combined groups.

### DETAILED DESCRIPTION

As used herein and in the appended claims, the singular forms “a,” “an,” and “the” include plural referents unless the context clearly dictates otherwise. Thus, for example, reference to an “antibody” includes a plurality of antibodies and reference to the “lipoprotein (a)” includes reference to one or more lipoprotein (a) s and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that any reference is prior art. Moreover, with respect to any term that is presented in one or more publications that is similar to, or identical with, a term that has been expressly defined in this disclosure, the definition of the term as expressly provided in this disclosure will control in all respects.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Lipoprotein (a) (Lp(a)) is common in the human population. Lp(a) is composed of apolipoprotein (a) (apo (a)) covalently bound to the apolipoprotein B-100 (apoB) moiety of LDL. Like LDL, Lp(a) contains cholesterol esters, free cholesterol, phospholipids, triglycerides and carbohydrates on its apolipoprotein components. While “LDL-C”, obtained by beta-quantification, Friedewald or Martin-Hopkins calculations, or direct LDL-C assays, has been generally accepted as an accurate biomarker for LDL-mediated CVD risk; “LDL-C” is actually a composite measurement of the cholesterol content on LDL, Lp(a), and IDL particles. Almost all patients have circulating Lp(a), there is enourmous interindividual heterogenetity in Lp(a) levels (>1,000 fold differences in the population), and approximately 20% of the population have highly elevated levels >50 mg/dL. Lp(a) and LDL have distinct biological activities, each mediating CVD risk independently. Moreover, LDL and Lp(a) respond differently to lipid lowering therapies, with statins causing a rise in Lp(a) compared to a decrease in LDL.

It had been generally accepted that the cholesterol content of Lp(a) is 30-45%. However, it is important to note that this estimation was based on a small number of studies that had biochemically characterized Lp(a) purified from only 3-4 individuals in each study. These studies had been performed prior to the advent of contemporary immunologic methods of Lp(a) mass measurement and may not necessarily be translatable for calculation of Lp(a)-C based on Lp(a) mass assayed in mg/dL using current assays.

As used herein “LDL-C” refers to the total cholesterol content in a plasma sample.

As used herein “corrected LDL-C” refers to the amount of cholesterol in a sample (e.g., a plasma sample) that lacks Lp(a)-cholesterol.

As used herein “Lp(a)-C” refers to the cholesterol content in an Lp(a) isolated fraction of plasma.

The disclosure provides methods and compositions for quantifying Lp(a)-C that are useful in determining mass/cholesterol relationships in subjects and provides a more accurate report of LDL-C. Moreover, the compositions and methods of the disclosure provide additional information for therapeutic interventions that affect cholesterol lowering.

In order to more accurately understand an individual's LDL-C attributable risk and to more accurately monitor treatment effects on LDL and Lp(a) individually, correct LDL-C without its Lp(a)-C component needs to be quantified. The disclosure provides a sensitive, high-throughput, and rapid assay to measure Lp(a)-C, which can complement the traditional lipid profile for determination of LDL-C.

The disclosure provides a rapid, high throughput, specific and sensitive assay to quantify Lp(a)-C. The methods and compositions of the disclosure provide for the following observations: (1) Lp(a)-C can be accurately measured in subjects with plasma Lp(a) levels up to 99th percentile of population levels; (2) the accepted percent of Lp(a) cholesterol relative to its mass, previously reported as 30%, is more variable than previously reported with a range of 6-57% among individuals, accordingly the disclosure provides a more accurate measurement; (3) the contribution of Lp(a)-C to LDL-C can be substantial and clinically relevant, with an average of 17 mg/dL in subjects with elevated Lp(a), which can translate to 10% difference in relative risk based on therapeutic studies; (4) subjects with substantially elevated Lp(a) have significantly lower correct LDL-C than appreciated; (5) direct LDL assays also measure Lp(a)-C in proportion to the amount of Lp(a) present in the sample.

The methods, compositions and findings provided herein have several important implications for clinical care, including assessing the role of Lp(a)-C and corrected LDL-C in risk prediction, reclassifying LDL-C thresholds in clinical diagnosis, and assessing treatment effects. Conventional lipid lowering therapies such as statins do not lower Lp(a) and may increase it. With the advent of highly effective Lp(a) lowering therapies, understanding an individual's correct LDL-C can guide the choice of the appropriate intensity and combination of therapies required to achieve guideline directed “LDL-C” goals.

As the absence of plasma Lp(a) is rare, all “LDL-C” assays do not accurately reflect the correct LDL-C. To date, the inclusion of Lp(a)-C in “LDL-C” measurements stems from the inability to separate Lp(a) from LDL, due to shared composition and overlapping densities. Clinical assays are referenced to beta-quantification, which shares this limitation. Therefore, “LDL-C” calculated by Friedewald will also inaccurately reflect LDL-C. The Martin-Hopkins formula is an advance over Friedewald, but suffers from the same limitations in being referenced to beta-quantitation, which includes the Lp(a)-C content. As shown here, direct LDL-C assays have the same limitation in measuring 84-98% of the cholesterol content on Lp(a). While the inaccuracy of “LDL-C” may be negligible in individuals with low Lp(a) levels, it can be significant in individuals with elevated Lp(a), who are common in the population. With the technique reported here, traditional reporting of “LDL-C” can be complemented by directly measured Lp(a)-C, and LDL-Ccorr (“LDL-C” minus Lp(a)-C) determined as a more accurate reflection of correct LDL-C. Alternatively, Lp(a) immuno-depleted plasma, which can be accomplished using an antibody or other binding domain that binds Lp(a) conjugated to magnetic beads (e.g., LPA4 antibody-beads) without any change in plasma volume (therefore preserving its non-Lp(a) component concentrations), can be assayed for correct LDL-C using conventional assays such as a direct LDL-C assay or by Friedewald or Martin Hopkins calculation.

The Lp(a)-C assay described here has been validated with spike-in experiments with purified Lp(a). The Lp(a)-C assay is linear across a range of about 2.9-747.0 nM Lp(a) input, although there was an about 20-26% (or less) overestimation bias with Lp(a) levels of 5.8 nM or less, which are not clinically important. The assay is suitable for the majority of the population, even those with Lp(a) mass levels at the 99th percentile. Importantly, those with elevated Lp(a) mass would more likely have a significant component of “LDL-C” as Lp(a)-C. The Lp(a)-C assay is specific, and does not detect cholesterol in plasma from transgenic mice expressing human LDL, along with endogenous lipoproteins, but lack Lp(a). Lp(a)-C correlated well with Lp(a) molar concentrations, further supporting the high specificity of this assay.

The Lp(a)-C assay of the disclosure is high-throughput and can be performed on 96-well plates or adapted for use with existing clinical analyzers using magnetic beads, microfluidics and the like. The entire assay is completed within about 1 hour. Other Lp(a)-C assays have been described, including those using electrophoretic, single density gradient ultracentrifugation, and affinity based on porous matrices using wheat germ agglutinin (which can bind glycoproteins such as apo (a)) or polyclonal anti-Lp(a) serum. However, no gold-standard assay nor reference materials to standardize or harmonize Lp(a)-C assays currently exist.

The disclosure demonstrates that the % Lp(a)-C can vary 10-fold in plasma from individuals with elevated Lp(a) who have had serial blood sampling as part of an ASO mediated Lp(a) lowering clinical trial. Across time points, % Lp(a)-C was not statistically different and the coefficient of variation was 20.38, suggesting that the variation is due to inter-individual and not treatment related differences. It is also of note that the Lp(a) mass assays in mg/dl, used as a denominator for % Lp(a)-C, are flawed. Although it is implied that the protein, lipid, and carbohydrate components of Lp(a) are measured, in reality only the apo (a) component is detected immunologically. Relative units corresponding to the amount of apo (a) detected are converted to mg/dL values based on calibrators with mg/dL values assigned to them in a non-standardized manner. Because Lp(a) mass is highly heterogenous between individuals, not only due to differences in apo (a) isoform size, but multiple variables such as glycosylation on apo (a) and lipid content, there is no primary reference material for standardization of Lp(a) measurement in mg/dL. A recent NHLBI working group for Lp(a) has recommended against using mg/dL assays for Lp(a) measurement. Therefore, estimation of Lp(a)-C based on a fixed assumed percent cholesterol content of Lp(a) mass in mg/dL may be a currently “expedient” first step estimate, but will not be accurate for most individuals and not optimally informative of the importance of Lp(a)-C as a risk factor or its response to therapy. For more precise CVD risk assessment and management, directly measured Lp(a)-C or measurement of LDL-C in Lp(a) immuno-depleted plasma will be necessary.

In certain embodiments of the disclosure antibody, antibody fragments or immunoglobulins are used to bind apo (a) thereby complexing with Lp(a). The terms “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and may also include certain antibody fragments. An antibody can be human, humanized and/or affinity matured. One antibody useful in the methods and compositions of the disclosure is the LPA4 antibody. The LPA4 antibody is a murine monoclonal antibody (Tsimikas et al., Circulation. 2004; 109:3164-3170). Another antibody useful in the methods and compositions of the disclosure is an antibody having the characteristics as set forth in Table A (see also International Application No. PCT/US2021/029307, incorporated herein by reference for all purposes):

TABLE A
LPA-KIV9 Heavy chain (IGHV1-15*01) (polynucleotide sequence = SEQ ID
NO: 1; polypeptide sequence = SEQ ID NO: 2; CDR1 = SEQ ID NO: 3 and 4;
CDR2 = SEQ ID NO: 5 and 6; CDR3 = SEQ ID NO: 7 and 8)

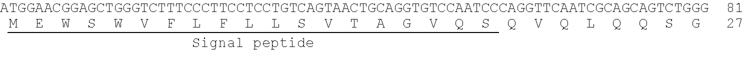

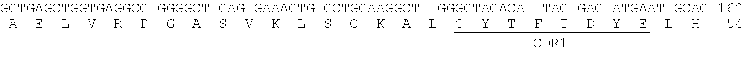

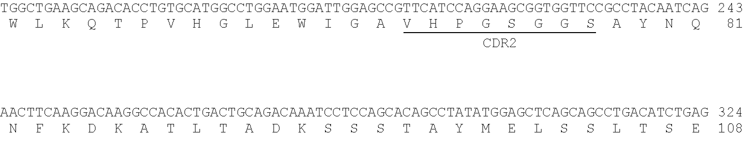

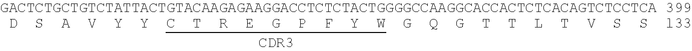

LPA-KIV9 Light chain (IGKV1-110*01) (polynucleotide sequence = SEQ
ID NO: 9; polypeptide sequence = SEQ ID NO: 10; CDR1 = SEQ ID NO: 11 and
12; CDR2 = SEQ ID NO: 13 and 14; CDR3 = SEQ ID NO: 15 and 16)

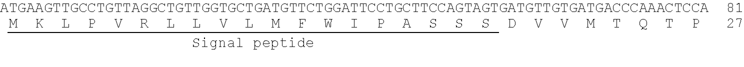

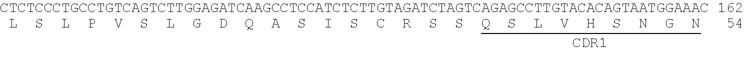

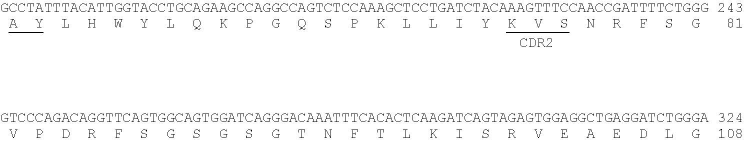

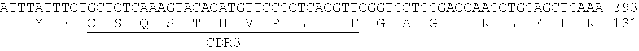

Other antibodies that bind to Lp(a) are known in the art and include both monoclonal, polyclonal and antibody fragments. These antibodies, as well as LPA4 and an antibody (or fragment thereof) of Table A, can be used in the methods and compositions of the disclosure.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called a, b, c, y, and u, respectively. The subunit structures and three-dimensional configurations of different classes of immunoglobulins are known.

“Antibody fragments” comprise only a portion of an intact antibody, wherein the portion typically retains at least one, more commonly most, or all, of the functions normally associated with that portion of the antibody when present in an intact antibody. Examples of antibody fragments include Fab, Fab′, F(ab′) 2, and Fv fragments; diabodies; linear antibodies; single-chain antibody molecules (scFv); and multispecific antibodies formed from antibody fragments. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcR binding, antibody half-life modulation, ADCC function and complement binding. In one embodiment, an antibody fragment is a monovalent antibody that has an in vivo half-life substantially similar to an intact antibody. For example, such an antibody fragment may comprise on antigen binding arm linked to an Fc sequence capable of conferring in vivo stability to the fragment.

An “antigen” is a target to which an antibody can selectively bind. The target antigen may be a polypeptide, a carbohydrate, a nucleic acid, a lipid, a hapten, a small molecule, or other naturally occurring or synthetic compound. In one embodiment of the disclosure an antigen is Lp(a). In another embodiment, the anybody binds to the antigen at an epitope contained in the sequence 4068CSETESGVLETPTVVPVPSMEAH4090 (SEQ ID NO: 17).

The term “array,” as used herein, generally refers to a predetermined spatial arrangement of binding islands, biomolecules, or spatial arrangements of binding islands or biomolecules. Arrays according to the disclosure that include biomolecules immobilized on a surface may also be referred to as “biomolecule arrays.” Arrays according to the disclosure that comprise surfaces activated, adapted, prepared, or modified to facilitate the binding of biomolecules to the surface may also be referred to as “binding arrays.” Further, the term “array” may be used herein to refer to multiple arrays arranged on a surface, such as would be the case where a surface bore multiple copies of an array. Such surfaces bearing multiple arrays may also be referred to as “multiple arrays” or “repeating arrays.” The use of the term “array” herein may encompass biomolecule arrays, binding arrays, multiple arrays, and any combination thereof; the appropriate meaning will be apparent from context. The biological sample can include fluid or solid samples from any tissue of the body including plasma. The binding islands or biomolecules can be an antibody or other immunoglobulin-like molecule that specifically binds to apolipoprotein (a).

An array of the disclosure comprises a substrate. By “substrate” or “solid support” or other grammatical equivalents, herein is meant any material appropriate for the attachment of biomolecules and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TEFLON, etc.), polysaccharides, nylon or nitrocellulose, resins, silica or silica-based materials including silicon and modified silicon, carbon, metals, inorganic glasses, plastics, ceramics, and a variety of other polymers. In addition, as is known the art, the substrate may be coated with any number of materials, including polymers, such as dextrans, acrylamides, gelatins or agarose. Such coatings can facilitate the use of the array with a biological sample; including those derived from serum.

A planar array of the disclosure will generally contain addressable locations (e.g., “pads”, “addresses,” or “micro-locations”) of biomolecules in an array format. The size of the array will depend on the composition and end use of the array. Arrays containing from about 2 different biomolecules to many thousands can be made. In some embodiments, the compositions of the disclosure may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates. In some embodiments, the substrate comprises a lawn of biomolecules.

As an alternative to planar arrays, bead based assays in combination with flow cytometry or microfluidic systems have been developed to perform multiparametric immunoassays. In bead based assay systems the biomolecules (e.g., an antibody or fragment) can be immobilized on microspheres. Each biomolecule for each individual immunoassay can be coupled to a distinct type of microsphere (i.e., “microbead”) and the immunoassay reaction takes place on the surface of the microspheres. Dyed microspheres with discrete fluorescence intensities are loaded separately with their appropriate biomolecules. The different bead sets carrying different capture probes can be pooled as necessary to generate custom bead arrays. Bead arrays are then incubated with the sample in a single reaction vessel to perform the immunoassay. The beads can be magnetic such that they can be actively manipulated through a microfluidic system or other fluidic separation system using magnets.

The term “anti-Lp(a) antibody” or “an antibody that binds to Lp(a)” refers to an antibody that is capable of binding Lp(a) with sufficient affinity such that the antibody is useful as a diagnostic and/or therapeutic agent in targeting Lp(a) or removing (e.g., panning) Lp(a) from a sample. In some embodiments of the disclosure an anti-Lp(a) antibody binds specifically to KIV9 and KIV2 without binding to plasminogen. In a specific embodiment, the antibody or antibody fragment binds to an epitope contained in the sequence 4068CSETESGVLETPTVVPVPSMEAH4090 (SEQ ID NO: 17).

“Binding affinity” generally refers to the strength of the sum total of non-covalent interactions between a binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule ‘X’ for its partner ‘Y’ can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the disclosure.

A “biological sample” encompasses a variety of sample types obtained from an individual and can be used in a diagnostic or monitoring assay. The definition encompasses blood, plasma, serum, sputum, cerebral spinal fluid, urine and other liquid samples of biological origin; or tissue cultures and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The biological sample may contain compounds which are not naturally intermixed with the tissue in nature such as preservatives, anticoagulants, buffers, fixatives, nutrients, antibiotics, or the like.

A “disorder” or “disease” is any condition that would benefit from diagnosis with a substance/molecule or method of the disclosure. This includes chronic and acute disorders or diseases including those pathological conditions which predispose the mammal to the disorder in question. Non-limiting examples of disorders to be treated herein include cardiovascular diseases and disorders.

A “cardiovascular disease” is a disease characterized by clinical events including clinical symptoms and clinical signs. Clinical symptoms are those experiences reported by a patient that indicate to the clinician the presence of pathology. Clinical signs are those objective findings on physical or laboratory examination that indicate to the clinician the presence of pathology. “Cardiovascular disease” includes both “coronary artery disease” and “peripheral vascular disease.” Clinical symptoms in cardiovascular disease include chest pain, shortness of breath, weakness, fainting spells, alterations in consciousness, extremity pain, paroxysmal nocturnal dyspnea, transient ischemic attacks and other such phenomena experienced by the patient. Clinical signs in cardiovascular disease include such findings as EKG abnormalities, altered peripheral pulses, arterial bruits, abnormal heart sounds, rales and wheezes, jugular venous distention, neurological alterations and other such findings discerned by the clinician. Clinical symptoms and clinical signs can combine in a cardiovascular disease such as a myocardial infarction (MI) or a stroke (also termed a “cerebrovascular accident” or “CVA”), where the patient will report certain phenomena (symptoms) and the clinician will perceive other phenomena (signs) all indicative of an underlying pathology. “Cardiovascular disease” includes those diseases related to the cardiovascular disorders of fragile plaque disorder, occlusive disorder and stenosis. For example, a cardiovascular disease resulting from a fragile plaque disorder, as that term is defined below, can be termed a “fragile plaque disease.” Clinical events associated with fragile plaque disease include those signs and symptoms where the rupture of a fragile plaque with subsequent acute thrombosis or with distal embolization are hallmarks. Examples of fragile plaque disease include certain strokes and myocardial infarctions. As another example, a cardiovascular disease resulting from an occlusive disorder can be termed an “occlusive disease.” Clinical events associated with occlusive disease include those signs and symptoms where the progressive occlusion of an artery affects the amount of circulation that reaches a target tissue. Progressive arterial occlusion may result in progressive ischemia that may ultimately progress to tissue death if the amount of circulation is insufficient to maintain the tissues. Signs and symptoms of occlusive disease include claudication, rest pain, angina, and gangrene, as well as physical and laboratory findings indicative of vessel stenosis and decreased distal perfusion. As yet another example, a cardiovascular disease resulting from restenosis can be termed an in-stent stenosis disease. In-stent stenosis disease includes the signs and symptoms resulting from the progressive blockage of an arterial stent that has been positioned as part of a procedure like a percutaneous transluminal angioplasty, where the presence of the stent is intended to help hold the vessel in its newly expanded configuration. The clinical events that accompany in-stent stenosis disease are those attributable to the restenosis of the reconstructed artery.

A “cardiovascular disorder” refers broadly to both coronary artery disorders and peripheral arterial disorders. The term “cardiovascular disorder” can apply to any abnormality of an artery, whether structural, histological, biochemical or any other abnormality. This term includes those disorders characterized by fragile plaque (termed herein “fragile plaque disorders”), those disorders characterized by vaso-occlusion (termed herein “occlusive disorders”), and those disorders characterized by restenosis. A “cardiovascular disorder” can occur in an artery primarily, that is, prior to any medical or surgical intervention. Primary cardiovascular disorders include, among others, atherosclerosis, arterial occlusion, aneurysm formation and thrombosis. A “cardiovascular disorder” can occur in an artery secondarily, that is, following a medical or surgical intervention. Secondary cardiovascular disorders include, among others, post-traumatic aneurysm formation, restenosis, and post-operative graft occlusion.

A “coronary artery disease” (“CAD”) refers to a vascular disorder relating to the blockage of arteries serving the heart. Blockage can occur suddenly, by mechanisms such as plaque rupture or embolization. Blockage can occur progressively, with narrowing of the artery via myointimal hyperplasia and plaque formation. Those clinical signs and symptoms resulting from the blockage of arteries serving the heart are manifestations of coronary artery disease. Manifestations of coronary artery disease include angina, ischemia, myocardial infarction, cardiomyopathy, congestive heart failure, arrhythmias and aneurysm formation. It is understood that fragile plaque disease in the coronary circulation is associated with arterial thrombosis or distal embolization that manifests itself as a myocardial infarction. It is understood that occlusive disease in the coronary circulation is associated with arterial stenosis accompanied by anginal symptoms, a condition commonly treated with pharmacological interventions and with angioplasty.

“Framework” or “FR” residues are those variable domain residues of an antibody or immunoglobulin other than the HVR (sometimes referred to as “CDR”) residues as herein defined.

The term “hypervariable region,” “HVR,” or “HV,” (sometimes referred to as CDRs) when used herein refers to the regions of an antibody variable domain which are hypervariable in sequence and/or form structurally defined loops. Generally, antibodies comprise six HVRs (CDRs); three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

An “individual,” “subject,” or “patient” is a vertebrate. In certain embodiments, the vertebrate is a mammal. Mammals include, but are not limited to, farm animals (such as cows), sport animals, pets/companion animals (such as cats, dogs, and horses), primates, mice and rats. In certain embodiments, a mammal is a human.

An “isolated” antibody, antibody fragment, polypeptide, antigen and the like refer to an antibody etc., which has been identified and separated and/or recovered from a component of its natural environment. Contaminant components of its natural environment include materials which would interfere with diagnostic or therapeutic uses for the antibody or antibody fragment, and may include enzymes, hormones, and other proteinaceous or nonproteinaceous solutes. In some embodiments, the antibody, antibody fragment etc. is purified (1) to greater than 95% by weight as determined by the Lowry method, and typically more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. An isolated antibody or antibody fragment includes the antibody or antibody fragment in situ within recombinant cells since at least one component of the antibody's natural environment is not present. Ordinarily, however, an isolated antibody or antibody fragment is prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The word “label” when used herein refers to a compound or composition which is conjugated or fused directly or indirectly to a reagent such as a nucleic acid probe or an antibody or antibody fragment and facilitates detection of the reagent to which it is conjugated or fused. The label may itself be detectable (e.g., radioisotope labels, luminescent or fluorescent labels) or, in the case of an enzymatic label, may catalyze chemical alteration of a substrate compound or composition which is detectable.

The “light chains” of antibodies (immunoglobulins) from any vertebrate species can be assigned to one of two clearly distinct types, called kappa (κ) and lambda (λ), based on the amino acid sequences of their constant domains.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier term “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody for purposes of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

“Lipoprotein (a)” or “Lp(a)” refer to a low-density lipoprotein variant containing a protein called apolipoprotein (a) (Apo (a)). Genetic and epidemiological studies have identified lipoprotein (a) as a risk factor for atherosclerosis and related diseases, such as coronary heart disease and stroke. Lipoprotein (a) consists of an LDL-like particle and the specific apolipoprotein (a), which is bound covalently to the apoB contained in the outer shell of the particle. Apo (a) proteins vary in size due to a size polymorphism (KIV-2 VNTR), which is caused by a variable number of kringle IV repeats in the LPA gene. This size variation at the gene level is expressed on the protein level as well, resulting in apo (a) proteins with 10 to more than 50 kringle IV repeats (each of the variable kringle IV consists of 114 amino acids). These variable apo (a) sizes are known as “apo (a) isoforms”. There is a general inverse correlation between the size of the apo (a) isoform and the Lp(a) plasma concentration. Lp(a) concentrations can vary by more than one thousand between individuals, from <0.2 to >200 mg/dL. There is a two- to three-fold higher mean Lp(a) plasma concentration in populations of African descent compared to Asian, Oceanic, or European populations. The general inverse correlation between apo (a) isoform size and Lp(a) plasma concentration is observed in all populations.

Lp(a) contributes to the process of atherogenesis. The structure of apolipoprotein (a) is similar to plasminogen and tPA (tissue plasminogen activator) and it competes with plasminogen for its binding site, leading to reduced fibrinolysis. Also, because Lp(a) stimulates secretion of PAI-1, it leads to thrombogenesis. It also may enhance coagulation by inhibiting the function of tissue factor pathway inhibitor. Moreover, Lp(a) carries atherosclerosis-causing cholesterol and binds atherogenic pro-inflammatory oxidised phospholipids as a preferential carrier of oxidised phospholipids in human plasma, which attracts inflammatory cells to vessel walls and leads to smooth muscle cell proliferation. Moreover, Lp(a) also is hypothesised to be involved in wound healing and tissue repair by interacting with components of the vascular wall and extracellular matrix. Apo (a), a distinct feature of the Lp(a) particle, binds to immobilized fibronectin thereby providing Lp(a) with the serine-proteinase-type proteolytic activity.

High Lp(a) in blood correlates with coronary heart disease (CHD), cardiovascular disease (CVD), atherosclerosis, thrombosis, and stroke. High Lp(a) correlates with early atherosclerosis independently of other cardiac risk factors, including LDL. In patients with advanced cardiovascular disease, Lp(a) indicates a coagulant risk of plaque thrombosis. Apo (a) contains domains that are very similar to plasminogen (PLG). Lp(a) accumulates in the vessel wall and inhibits the binding of PLG to the cell surface, reducing plasmin generation, which increases clotting. This inhibition of PLG by Lp(a) also promotes the proliferation of smooth muscle cells. These unique features of Lp(a) suggest that Lp(a) causes generation of clots and atherosclerosis. Numerous studies confirming a strong correlation between elevated Lp(a) and heart disease have led to the consensus that Lp(a) is an important independent predictor of cardiovascular disease. Animal studies have shown that Lp(a) may directly contribute to atherosclerotic damage by increasing plaque size, inflammation, instability, and smooth muscle cell growth. Genetic data also support the theory that Lp(a) causes cardiovascular disease. Lp(a) is associated with enhanced atherogenic potential, particularly at levels >30 mg/dl, and is shown to be an independent predictor (odds ratio-1.5-2) of cardiovascular risk, particularly in younger subjects (<60 years old) and those with elevated LDL cholesterol levels.

Lp(a) appears with different isoforms (per kringle repeats) of apolipoprotein; 40% of the variation in Lp(a) levels when measured in mg/dl can be attributed to different isoforms. Lighter Lp(a) are also associated with disease. Thus, a test with simple quantitative results may not provide a complete assessment of risk.

“PAD” or “peripheral artery disease” encompasses disease states such as atherosclerosis and atherothrombosis that occur outside the heart and brain. It is a common comorbid disease with CAD. Subjects who are deemed to be at low risk or no risk of PAD based upon an assessment of traditional risk factors of PAD (or arteriovascular disease), or who are asymptomatic for PAD or an arteriovascular disease may nevertheless be at risk for an arteriovascular event, even in the absence of claudication. Claudication can be defined as pain or discomfort in the muscles of the legs occurring due to a decreased amount of blood flowing to a muscle from narrowing of the peripheral arteries, producing ischemia and often arterial occlusion, causing skeletal muscle and limb necrosis. The pain or discomfort often occurs when walking and dissipates under resting conditions (intermittent claudication). Pain, tightness, cramping, tiredness or weakness is often experienced as a result of claudication. PAD not only causes the hemodynamic alterations common in CAD, but also results in metabolic changes in skeletal muscle. When PAD has progressed to severe chronic and acute peripheral arterial occlusion, surgery and limb amputation often become the sole therapeutic options. PAD is widely considered to be an underdiagnosed disease, with the majority of confirmed diagnoses occurring only after symptoms are manifested, or only with other arteriovascular disease, and irreversible arteriovascular damage due to such ischemic events has already occurred.

In the context of the disclosure, “population” refers to any selected group of individuals, such as individuals that live in a particular geographic region, country or state. In some cases, the population is a group of subjects, such as a group of subjects that participated in a clinical study. In another embodiment, a population can comprise an ethnic group, an age group or can be based on sex. In particular examples, the population is the Framingham Offspring Cohort.

The term “propensity to disease,” also “predisposition” or “susceptibility” to disease or any similar phrase, means that certain markers are associated with or predictive of a subject's incidence of developing a particular disease (herein, a cardiovascular disease). The biomarker (e.g., the presence of a particular ratio or level of phospholipid or apoprotein) are thus over-represented or underexpressed (depending upon the marker) in frequency in individuals with disease as compared to healthy individuals.

A “risk factor” is a factor identified to be associated with an increased risk. A risk factor for a stroke or cardiovascular disorder or a cardiovascular disease is any factor identified to be associated with an increased risk of developing those conditions or of worsening those conditions. A risk factor can also be associated with an increased risk of an adverse clinical event or an adverse clinical outcome in a patient with a cardiovascular disorder. Risk factors for cardiovascular disease include smoking, adverse lipid profiles, elevated lipids or cholesterol, diabetes, hypertension, hypercoagulable states, elevated homocysteine levels, and lack of exercise. Carrying a particular polymorphic allele is a risk factor for a particular cardiovascular disorder, and is associated with an increased risk of the particular disorder.

The term “substantially similar” or “substantially the same,” as used herein, denotes a sufficiently high degree of similarity between two numeric values (for example, one associated with an antibody of the disclosure and the other associated with a reference/comparator antibody), such that one of skill in the art would consider the difference between the two values to be of little or no biological and/or statistical significance within the context of the biological characteristic measured by the values (e.g., K_{d} values). The difference between said two values is, for example, less than about 508, less than about 408, less than about 308, less than about 208, and/or less than about 108 as a function of the reference/comparator value.

The phrase “substantially reduced,” “substantially increased,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{d} values). The difference between said two values is, for example, greater than about 10%, greater than about 20%, greater than about 30%, greater than about 408, and/or greater than about 50% as a function of the value for the reference/comparator molecule.

In one embodiment, of the disclosure a method for determining a subject's Lp(a)-cholesterol (Lp(a)-C) amounts and/or LDL-C amounts and/or predisposition to coronary artery disease is provided. In one embodiment, the disclosure provides a method of determining Lp(a)-C from a plasma sample, the method comprising contacting a sample with a binding agent that specifically binds to apo (a) isolating the resulting binding agent-apo (a) complex and measuring the amount of cholesterol in the isolated complex to obtain and Lp(a)-C value. This method can be performed on a sample or subsample. In one embodiment, a matching subsample undergoes analysis of total LDL cholesterol present in the sample (LDL-C).

In another embodiment, the method comprises separating Lp(a)-C from a plasma sample to obtain and Lp(a)-C subsample and a Lp(a)-free subsample, and measuring the amount of LDL-cholesterol in the Lp(a)-free subsample. In a further embodiment, the method includes measuring the amount of cholesterol in the Lp(a)-C subsample. In a further embodiment, the amount of cholesterol in each subsample or the combine values are used to determine a subject's risk of CVD.

In yet another embodiment, the method comprises measuring the total LDL-cholesterol in a plasma sample, removing Lp(a)-C from the plasma sample, measuring the amount of Lp(a)-cholesterol to obtain an Lp(a)-C value, and subtracting that amount from the total LDL-cholesterol value to obtain a corrected LDL-C value.

In another embodiment, the disclosure provides a method of determining whether treatment for LDL-cholesterol is working, the method comprising measuring LDL-cholesterol, Lp(a)-cholesterol or a corrected LDL-cholesterol (e.g., the amount of LDL-C in a sample that has been treated to remove Lp(a)) prior to LDL-cholesterol therapy, during therapy or following therapy, wherein a reduction in corrected LDL-C is indicative of an effective therapy.

The LDL-cholesterol values obtained by the methods of the disclosure are more accurate than the existing methods and can be used to correlate a risk or disease status to the subject. The Lp(a) can be detected/removed from a sample using an antibody, antibody fragment or binding domain that specifically binds to an Lp(a).

The method can be carried out on a biological sample obtained from a subject. The biological sample can be, for example, blood, serum, or plasma.

In some embodiments, the antibody, antibody fragment or binding domain are immobilized on a substrate and in some embodiments are immobilized to form an array. In one embodiment, an immunoassay can be performed either by first capturing the Lp(a) on a microtiter well by use of an antibody of the disclosure, and then detecting the amount of cholesterol in the capture fraction and/or the Lp(a)-free fraction.

Anti-Lp(a) antibodies of the disclosure can be produced transgenically through the generation of a mammal or plant that is transgenic for the immunoglobulin heavy and light chain sequences of interest and production of the antibody in a recoverable form therefrom. In connection with the transgenic production in mammals, anti-Lp(a) antibodies can be produced in, and recovered from, the milk of goats, cows, or other mammals. See, e.g., U.S. Pat. Nos. 5,827,690, 5,756,687, 5, 750, 172, and 5,741, 957, incorporated herein by reference. Nucleotide and polypeptide sequences useful in the generation of anti-Lp(a) antibodies and fragments thereof are provided in Table A.

The disclosure also provides kits compartmentalized to include one or more reagents for carrying out the methods of the disclosure. Such kits can include ELISA-based assay systems comprising at least an antibody that binds to Lp(a). In some embodiments, the kit can comprise a micro-bead comprising an antibody and optionally a microfluidic system for assaying Lp(a)-C or LDL-C in a sample.

The following examples are intended to illustrate but not limit the disclosure. While they are typical of those that might be used, other procedures known to those skilled in the art may alternatively be used.

### EXAMPLES

### Example 1

Study population. Lp(a)-C was measured in a subset of subjects with elevated baseline Lp(a) (>125 nM) from a phase II, placebo controlled randomized trial of antisense oligonucleotide (ASO) mediated Lp(a) lowering. Plasma samples from 8 subjects who received placebo and 21 subjects who received Lp(a) lowering (IONIS-APO (a) Rx) ASO at baseline, peak treatment effect (day 85/99), and at the end of the study (day 190) when Lp(a) levels have recovered following ASO washout, were included in this analysis. This cohort was intentionally chosen to evaluate the specificity of the Lp(a)-C assay over a wide range of Lp(a), as Lp(a) molar concentrations decreased by an average of 63.8±19.5% (mean±S. D.), whereas total cholesterol, “LDL-C”, and HDL-C were not significantly changed with IONIS-APO (a) Rx ASO.

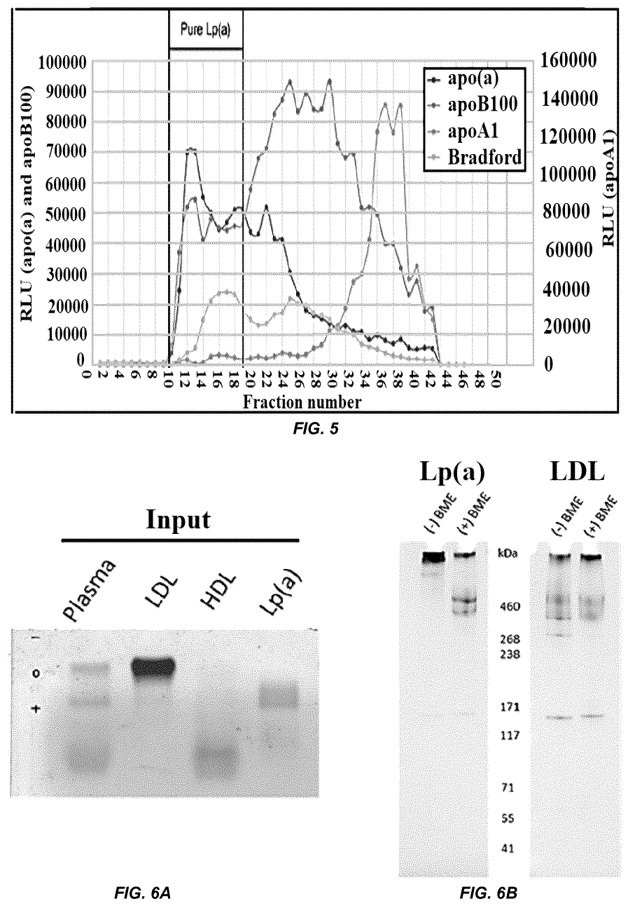

Lipoprotein (a) purification. Purified Lp(a) used in spike-in experiments was isolated from the Liposorber post-apheresis eluent from a single donor undergoing lipid apheresis for the treatment of homozygous familial hypercholesterolemia. This subject's pre-apheresis plasma Lp(a) level was 85 nM with a predominant (>95%) apo (a) isoform size of 24 kringle IV repeats. To prevent oxidation in storage, EDTA (2 mM) and beta-hydroxybutyrate (20 uM) final concentrations respectively were added to the eluent. To prevent non-specific association between Lp(a) and other lipoproteins, proline and epsilon aminocaproic acid (EACA) were added at a final concentration of 200 mM each. The density of the eluent was adjusted by addition of NaBr for sequential ultracentrifugation in a Type-50 Ti rotor (Beckman) for 16-24 hours at 10° C. at 50,000 rpm and the 1.063 g/mL<density <1.090 g/mL fractions were harvested. This fraction was applied to an SW-400 gel filtration column (General Electric), and eluted into 0.5 mL fractions. Each fraction was assayed for the presence of apo (a), apolipoprotein B-100, and apoAI by ELISA. The fractions containing apo (a) and apolipoprotein B-100 in proportion to apo (a), but not those containing apoAI, were pooled (FIG. 5), concentrated and buffer exchanged into PBS with 0.5 mM EDTA using Amicon centrifugal filter units (Milipore). Lp(a) purity was assessed by lipoprotein agarose gel electrophoresis and SDS PAGE (FIG. 6).

Apolipoprotein quantification in fractions eluted from gel filtration column for Lp(a) purification. Five microliters from each 0.5 mL fraction eluted from gel filtration column was diluted into 395 microliters of PBS in a 96 well plate. Fifty microliters of each diluted fraction were then coated onto ELISA plates overnight at 4C. Each plate was then washed, blocked with 18 BSA in PBS, then 1 μg/ml of following antibodies against the desired apolipoproteins were added to their respective plate: biotin-LPA4 for apo (a), biotin-goat anti-human apolipoprotein B-100 (Academy BioMed), and biotin-goat anti-human apolipoprotein AI (Academy BioMed). After unbound antibodies were washed off, apolipoproteins were quantified by neutral-avidin conjugated alkaline phosphatase and Lumiphos (Lumigen) generated relative light units (RLUs) as read on a BioTek plate reader.

Total protein from each fraction was quantified by Bradford protein assay (Pierce) using the manufacturer's protocol.

SDS PAGE and protein staining. For purified lipoproteins, 1 μg of protein per sample in SDS loading buffer without or without beta-mercaptoethanol (BME) was loaded on a 3-88 tris acetate polyacrylamide gel. For immunoprecipitation experiments, SDS loading buffer with and without BME was added to the beads containing immunoprecipitate, and one-third value was loaded onto a gel. A high molecular weight pre-stained protein ladder (HiMark, ThermoFisher) accompanied each gel. Electrophoresis was performed at 100V. Proteins were visualized with a luminescent stain (Lumitein, Biotium) on an UV transmitting imaging station (Kodak).

Lipoprotein electrophoresis. Plasma or purified lipoproteins were resolved on an agarose gel and stained for lipids as per the manufacturer's protocol (Helena Laboratories).

Total cholesterol and direct LDL-C assays. Total cholesterol measurements were performed using an enzymatic, colorimetric assay (Pointe Scientific) according to the manufacturer's protocol. Direct LDL-C assays are assumed to only measure cholesterol on LDL particles; however, these assays are referenced to beta-quantification, a method that cannot distinguish Lp(a)-C from LDL-C due to overlapping densities of LDL and Lp(a) particles. To assess whether these direct LDL-C assays also inadvertently quantitate the Lp(a)-C content of the samples, colorimetric direct LDL-C reagents were purchased from Roche (LDLC3, Cat. 07005717), Sekisui (direct LDL-C, Cat. 7120), and Wako (L-Type LDL-C, Cat. 993-00404). Cholesterol assays were performed in clear, flat bottom 96-well plates. Two microliters of human plasma, purified Lp(a) as noted above, and assay calibrator from Wako (Cat. 990-28011) containing 150 mg/dL LDL-C were added to 2 microliters of PBS for a total of 4 microliters of input for each assay. For spike-in experiments, 2 microliters of purified Lp(a) was added to 2 microliters of plasma as input. Incubation times, temperature, and absorbance wavelengths were performed according to the manufacturer's protocol. Absorbance was quantified on a BioTek Synergy plate reader.

Lp(a) ELISA, LPA isoforms and OxPL-apoB levels. Plasma Lp(a) molar concentration (nM) and mass (mg/dL) were measured by the Northwest Lipid Research Laboratories (NWLRL) and our assays, respectively. Measurement of oxidized phospholipids on apoB (OxPL-apoB) were described in Viney et al. Lancet, 388 (10057): 2239-2253, 2016. Total cholesterol (TC), “LDL-C”, HDL-C, triglycerides (TG), and apoB were measured using commercial assays. A previously validated sandwich ELISA using an anti-apoB-100 capture antibody and the monoclonal anti-apo (a) detection antibody, LPA4, was performed as previously described (Tsimikas et al., J. Clin. Lipidology, 12 (5): 1313-1323, 2018). LPA isoforms and OxPL-apoB levels were measured as previously described in Viney et al. (supra).

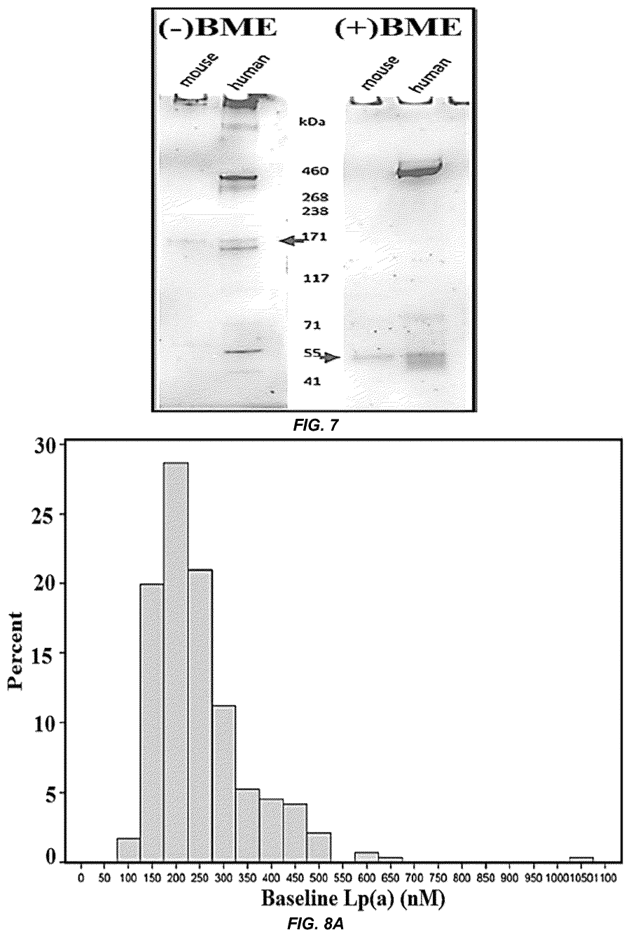

Generation of murine monoclonal antibody LPA4-coated magnetic beads. LPA4 was harvested from hybridomas expanded in mouse ascites and purified using a protein G affinity column (Abcore, Poway CA). Conjugation of LPA4 to dynabeads MyOne Epoxy magnetic beads (Life Technologies) was performed according to the manufacturer's protocol. Briefly, the coupling reaction involved 30 μg of LPA4 per milligram of dynabeads. Once coupled, unbound antibody was washed off according to the manufacturer's protocol and the LPA4 coupled dynabeads were stored in PBS. Specificity of LPA4-dynabead was assessed by immunoprecipitation from human plasma containing Lp(a) or mouse plasma lacking Lp(a), followed by SDS PAGE (FIG. 7).

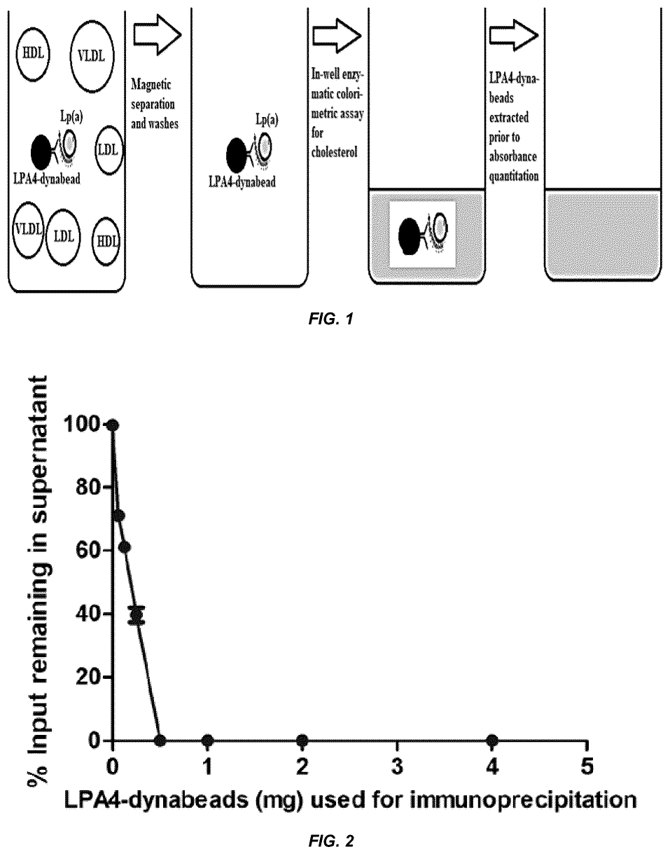

Measurement of Lp(a)-C. Fifteen microliters of each plasma sample were added to 15 ul of PBS containing 18 BSA, 200 mM proline and 200 mM EACA. The presence of proline and EACA prevents the association between Lp(a) and triglyceride rich lipoproteins. Each diluted plasma sample was assayed in duplicate and added to 1 mg of LPA4-dynabeads in U-bottom 96-well plate. The beads were resuspended on a plate shaker at 900 rpm for 10 seconds, followed by a 45-minute room temperature incubation with gentle shaking at 500 rpm to prevent the beads from precipitating. Lp(a) bound onto LPA4-dynabeads were then extracted from each well using a magnetic bead extraction replicator (VP Scientific, Cat. 407AM-N1) and released into a parallel 96-well plate containing 200 ul of PBS 18 BSA, 200 mM proline and 200 mM EACA in each well to wash off any non-specifically bound lipoproteins. This process was repeated for a total of 3 washes. Then LPA4-dynabeads containing Lp(a) were transferred to a parallel, clear, flat-bottom, 96-well plate containing 200 ul of enzymatic cholesterol reagent (Pointe Scientific), resuspended with shaking at 500 rpm, then incubated at 37° C. for 5 minutes. Each flat-bottom plate also contains a dedicated row that had been pre-populated with 2-fold serial dilutions of cholesterol standard (Pointe Scientific), ranging from 0.0375-1.5 ug cholesterol for generation of a standard curve. The plates were analyzed for absorbance at 500 nm (primary) and 700 nm (background). The amount (ug) of Lp(a)-C in each sample was determined based on the OD 500 nm-OD 700 nm value calibrated against the standard curve. Then, the concentration of Lp(a)-C was determined based on the input volume of plasma. In the case of a 15 ul input, the mg/dL of Lp(a)-C=ug Lp(a)×100/15. A simplified schematic of the Lp(a)-C assay is depicted in FIG. 1.

Determination of LDL-Ccorr. LDL-Ccorr was determined by subtracting the measured “LDL-C” from directly measured Lp(a)-C.

Mouse model. Transgenic mice expressing human apoB-100 only and human-like LDL, due to a mutation in codon 2153 preventing apoB-48 synthesis.

Statistics. Descriptive statistical analysis, correlation analysis using Spearman's rho test, analysis of variance between parametric datasets using ANOVA and non-parametric datasets using Kruskal-Wallis testing, was performed with SPSS version 26.

Immunoprecipitation of Lp(a) and direct quantification of its cholesterol content. Lp(a) was immunoprecipitated from plasma from a patient with an elevated Lp(a) particle concentration of 573 nM (value at the 99th percentile in the population) using a monoclonal antibody against apo (a), LPA4, directly coupled to non-porous magnetic beads (LPA4-dynabeads). One-half a milligram (0.5 mg) of LPA4-dynabeads was able to deplete all the Lp(a) from 15 ul of this plasma (FIG. 2). To accommodate for even higher Lp(a) levels in the population, and to accommodate a margin of error, all subsequent reactions described in this manuscript utilized 1 milligram of LPA4-dynabeads.

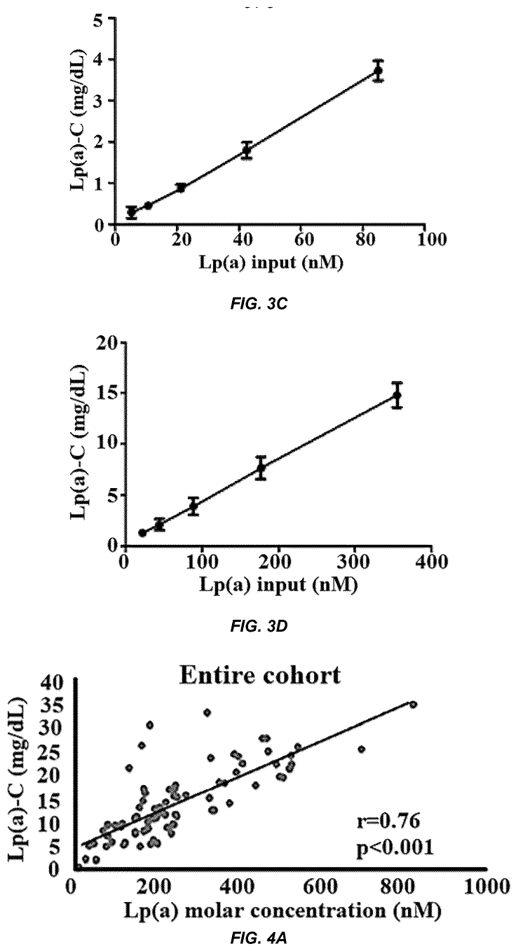

To test the specificity and linearity of cholesterol measured on LPA4-dynabead immunoprecipitated Lp(a) [Lp(a)-C], plasma from mice that do not express apo (a) or Lp(a) but express human-apolipoprotein B-100 (and therefore human-like LDL) with or without spiked-in purified Lp(a) were assayed for Lp(a)-C. Mice expressing human-apolipoprotein B-100 (hApoB) have a total cholesterol of 150 mg/dL, non-HDL-C of 120 mg/dl, and no circulating Lp(a), thus an Lp(a)-C of 0 mg/dL. Lp(a)-C was measured in hApoB mouse plasma with serial two-fold increments of purified Lp(a) spiked-in, ranging from 2.9 nM to 1494.0 nM (FIGS. 3A and B). There was a linear relationship between Lp(a)-C and the amount of spiked-in Lp(a) particle number up to 747.0 nM Lp(a), beyond which the assay is saturated (FIGS. 3A and B). Based on the total cholesterol of the spiked-in purified Lp(a) measured in parallel, the percent recovery (SD) was 126.3 (18.5) 8, 119.7 (14.0) %, 91.3 (0.5) %, 84.7 (5.5) %, 93.3 (10.2) %, 90.7 (5.5) %, 89.7 (9.5) %, 95.3 (7.2) %, and 94.3 (4.5) % with 2.9 nM, 5.8 nM, 11.7 nM, 23.3 nM, 46.7 nM, 93.4 nM, 186.8 nM, 373.5 nM, and 747.0 nM spiked-in purified Lp(a), respectively (FIG. 3B).

To further evaluate linearity of the assay, Lp(a)-C was determined in serial 2-fold dilutions of plasma from a patient with Lp(a) level of 85.0 nM (normal <75 nM) (FIG. 3C) and another patient with elevated Lp(a) of 355.0 nM (FIG. 3D). The R2 correlation coefficient between Lp(a)-C and Lp(a) particle concentration was 0.998 and 0.999, respectively.

Intra-assay coefficient of variation (CV) determined by 5 replicate Lp(a)-C measurements from plasma with Lp(a) particle concentrations of 2.7 nM, 12.2 nM, 26.2 nM, 51.9 nM, 54.4 nM, 165.8 nM, 181.9 nM, and 522.2 nM were 2.28, 7.78, 9.58, 4.68, 5.28, 1.0%, 5.6%, and 4.7%, respectively. Inter-assay CVs determined by Lp(a)-C measurements performed on 4 consecutive days from plasma with Lp(a) particle concentrations of 5.2 nM, 15.5 nM, 54.0 nM, 68.1 nM, 85.0 nM, 100.9 nM, and 390.0 nM were 0.88, 4.18, 10.08, 5.08, 12.0%, 6.0%, and 10.0%, respectively.

Lp(a)-C in a cohort of individuals with elevated plasma Lp(a) molar concentrations and following Lp(a) reduction with an antisense oligonucleotide. In this cohort, 21 patients received Lp(a) lowering therapy [IONIS-APO (a) px], with median (range) Lp(a) molar concentrations, as measured by the NWLRL assay, of 344. 6 (149.3-822.8) nM, 113.7 (9.0-505.8) nM, and 244.5 (67.2-697.0) nM at baseline, trough, and recovery timepoints, respectively (p Kruskal-Wallis <0.001) (Table 1). Lp(a) molar concentrations [mean (SD)] were 36.1 (19.5) % and 82.5 (29.5) % of baseline levels at trough and recovery timepoints, respectively (p ANOVA <0.001). In addition, for the current study, Lp(a) mass levels were also measured at each timepoint using the UCSD assay reporting data as total Lp(a) mass in mg/dL. At baseline, trough, and recovery, Lp(a) mass levels were 111.4 (55.3-157.8) mg/dl, 54.6 (2.8-103.2) mg/dl, and 87.0 (27.3-132.9) mg/dl, respectively (p Kruskal-Wallis <0.001). Plasma lipid parameters at each timepoint, including “LDL-C” determined by Friedewald calculation are described in Table 1. Median (range) Lp(a)-C levels measured in baseline, trough, and recovery timepoints were 14.2 (5.6-35.0) mg/dl, 7.4 (0.6-19.7) mg/dl, and 12.9 (5.5-25.7) mg/dl, respectively (p Kruskal-Wallis <0.001). For completeness, 8 individuals receiving placebo ASO were also evaluated. In this treatment group, none of the plasma lipid or Lp(a) parameters differed significantly between the 3 timepoints. The median (range) Lp(a) molar concentration, Lp(a) mass, and Lp(a)-C were 209.0 (131.9-542.4) nM, 73.9 (52.1-166.8) mg/dl, and 15.5 (8.4-30.8), respectively (Table 1) . . . 34

TABLE 1
Plasma lipoprotein parameters in 29 individuals with elevated baseline Lp(a) enrolled in a Lp(a) lowering ASO clinical trial

| | Placebo ASO (n = 8) | | IONIS-APO(a)_{Rx} ASO (n = 21) | | | | | |
|---|---|---|---|---|---|---|---|---|
| | All timepoints | | Baseline | | Trough | | Recovery | |
| TC [mean (SD)] (mg/dL) | 208 | (47.7) | 197.0 | (39.7) | 181.3 | (45.7) | 199.2 | (33.4) |
| “LDL-C” [mean (SD)] (mg/dL) | 122.1 | (28.0) | 116.3 | (36.8) | 102.2 | (41.7) | 118.1 | (34.6) |
| HDL-C [mean (SD)] (mg/dL) | 58.0 | (28.6) | 54.4 | (12.8) | 52.0 | (13.9) | 54.2 | (15.4) |
| TG [median (range)] (mg/dL) | 95.0 | (46.0-436.0) | 114.0 | (64.0-319.0) | 131.0 | (65.0-305.0) | 131.5 | (61.0-271.0) |
| apoB-100 [mean (SD)] (mg/dL) | 98.0 | (23.6) | 95.2 | (25.5) | 85.5 | (29.0) | 93.5 | (22.1) |
| Lp(a) molar concentration [median (range)] | 209.0 | (131.9-542.4) | 344.6 | (149.3-822.8)** | 113.7 | (9.0-505.8)** | 244.5 | (67.2-697.0)** |
| (nM) | | | | | | | | |
| Lp(a) mass [median (range)] (mg/dL) | 73.9 | (52.1-166.8) | 111.4 | (55.3-157.8)** | 54.6 | (2.8-103.2)** | 87.0 | (27.3-132.9)** |
| apo(a) major isoform [median (range)] | 16.0 | (14.0-20.0) | 17.0 | (13.0-19.0) | 17.0 | (13.0-19.0) | 17.0 | (13.0-19.0) |
| apo(a) minor isoform [median (range)] | 20.0 | (null-30.0) | 20.0 | (null-29.0) | 20.0 | (null-29.0) | 20.0 | (null-29.0) |
| OxPL-apoB [median (range)] (nM) | 21.3 | (13.6-37.2) | 25.2 | (16.8-41.2)** | 16.6 | (7.5-25.2)** | 22.0 | (12.3-37.3)** |
| Lp(a)-C (mg/dL) [median (range)] | 15.5 | (8.4-30.8) | 14.2 | (5.6-35.0)** | 7.4 | (0.6-19.7)** | 12.9 | (5.5-25.7)** |
| % Lp(a)-C [median (range)] | 17.9 | (9.6-57.3) | 15.6 | (6.9-41.5) | 18.7 | (5.8-45.4) | 16.0 | (8.4-31.4) |
| LDL-C_{corr} [median (range)] (mg/dL) | 98.5 | (65.1-165.4) | 95.2 | (38.0-162.6) | 90.3 | (33.9-214.9) | 110.5 | (47.5-169.0) |
| % Lp(a)-C/“LDL-C” [median (range)] | 11.3 | (7.2-25.0) | 15.0 | (5.4-43.4)* | 7.3 | (0.7-25.1)* | 11.8 | (5.2-28.1)* |

Values are reported as median (range). Baseline, trough, and recovery refers to timepoints associated with respective Lp(a) molar concentrations in IONIS-APO(a)_{Rx} ASO treated subjects. P Kruskal Wallis values comparing changes in subjects receiving apo(a)rx ASO across timepoints.
*Denotes p = 0.01;
**Denotes p < 0.001;
TC = total cholesterol;
TG = triglycerides;
LDL-C = low density lipoprotein cholesterol;
HDL-C = high density lipoprotein cholesterol;
apoB-100 = apolipoprotein B-100;
OxPL-apoB = oxidized phospholipid on apolipoprotein B-100;
Lp(a)-C = Lp(a) cholesterol;
% Lp(a)-C = Percent of Lp(a)-C/Lp(a) mass;
LDL-C_{corr} = “LDL-C” − Lp(a)-C;
% Lp(a)-C/“LDL-C” = Percent of Lp(a)-C/“LDL-C”

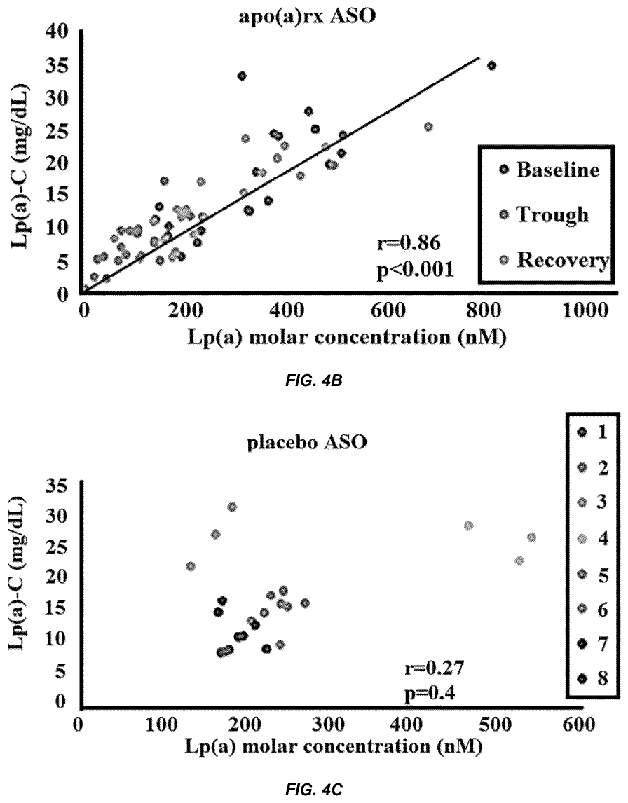

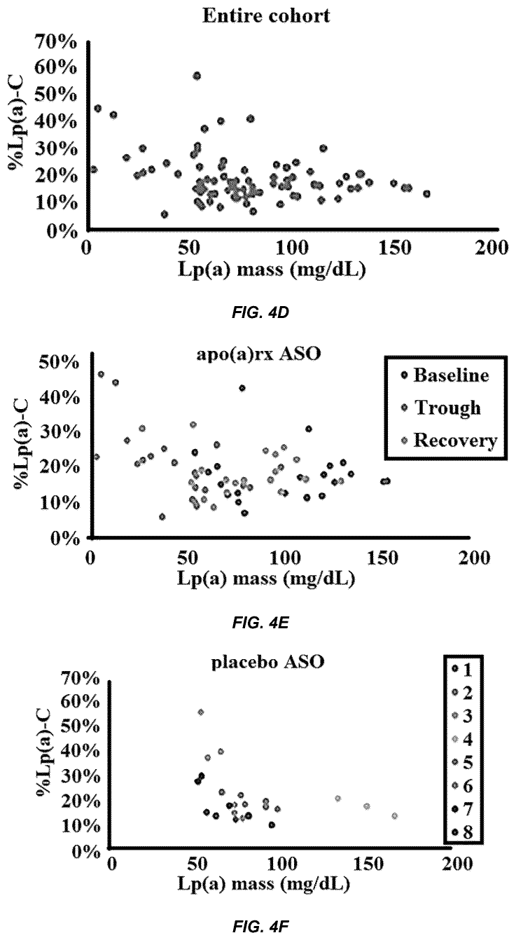

Relationship of Lp(a)-C to Lp(a) mass and concentration. In the entire cohort of 29 individuals over 3 time points, Lp(a)-C levels correlated well with Lp(a) molar concentration and mass measured in nM and mg/dL (FIG. 4A), with Spearman's rho (r) of 0.76 (p<0.001) and 0.69 (p<0.001), respectively (Table 2). Other statistically significant correlations with Lp(a)-C include OxPL-apoB (r=0.65, p<0.001), total cholesterol (r=0.39, p<0.001), and “LDL-C” (r=0.29, p=0.008). However, Lp(a)-C did not significantly correlate with LDL-Ccorr (r=0.10, p=0.35). When only the baseline ASO and placebo samples were analyzed together, the correlation between Lp(a)-C and Lp(a) molar concentration was 0.65, p<0.001.

TABLE 2
Spearman correlation coefficients (p-values) between Lp(a)-C and various
Lp(a) and plasma lipoprotein parameters in 29 individuals with elevated
baseline Lp(a) enrolled in a Lp(a) lowering ASO clinical trial

| | Lp(a) molar | | | | | |
|---|---|---|---|---|---|---|
| | (nM) | | Lp(a)-C (mg/dL) | | % Lp(a)-C | |
| Total cholesterol (mg/dL) | 0.24 | (p = 0.03) | 0.39 | (p < 0.001) | 0.36 | (p = 0.001) |
| “LDL-C” (mg/dL) | 0.25 | (p = 0.02) | 0.29 | (p = 0.008) | 0.25 | (p = 0.024) |
| LDL-C_{corr} (mg/dL) | 0.1 | (p = 0.4) | 0.1 | (p = 0.35) | 0.19 | (p = 0.09) |
| HDL-C (mg/dL) | 0.02 | (p = 0.9) | 0.05 | (p = 0.687) | 0.11 | (p = 0.320) |
| TG (mg/dL) | −0.06 | (p = 0.6) | 0.16 | (p = 0.162) | 0.28 | (p = 0.010) |
| Lp(a) mass (mg/dL) | 0.91 | (p < 0.001) | 0.69 | (p < 0.001) | −0.25 | (p = 0.023) |
| Lp(a) molar (nM) | 1 | | 0.76 | (p < 0.001) | −0.07 | (p = 0.541) |
| OxPL-apoB (nM) | 0.85 | (p < 0.001) | 0.65 | (p < 0.001) | 0.1 | (p = 0.104) |

| | | | | | |
|---|---|---|---|---|---|
| Lp(a)-C (mg/dL) | 0.76 | (p < 0.001) | 1 | 0.42 | (p < 0.001) |

| | | | | | |
|---|---|---|---|---|---|
| % Lp(a)-C | −0.07 | (p = 0.5) | 0.42 | (p < 0.001) | 1 |

Three timepoints (baseline, trough, and recovery) from each of the 29 individuals were included in this correlation analysis. OxPL-apoB = oxidized phospholipid on apolipoprotein B-100; TC = total cholesterol; LDL-C = low density lipoprotein cholesterol; HDL-C = high density lipoprotein cholesterol; TG = triglycerides; % Lp(a)-C = percent Lp(a)-C/Lp(a) mass. Statistically significant correlations in bold.

In baseline samples across the entire cohort, the proportion of “LDL-C” that was Lp(a)-C based on direct measurement was 13.2 (5.4-42.4) % [median (range)]. Baseline LDL-Ccorr was significantly lower than laboratory-measured “LDL-C” [mean (SD)]102.2 (31.8) vs 119.2 (32.4) mg/dl, respectively, with p<0.001.

To understand if the cholesterol content of Lp(a) varied between individuals, Lp(a)-C was expressed as a percentage of Lp(a) mass measured in mg/dl using the UCSD assay (Lp(a)-C). The median (range) % Lp(a)-C across the entire cohort was 17.3 (5.8-57.3) % (FIG. 4D). In individuals receiving IONIS-APO (a) Rx ASO, % Lp(a)-C was similar at baseline, trough, and recovery, 15.6 (6.9-41.5), 18.7 (5.8-45.4), and 16.0 (8.4-31.4), respectively (p Kruskal-Wallis=0.5) (FIG. 4E and Table 1). In the placebo group, Lp(a)-C was higher in those with lower Lp(a) mass, with spearman correlation r=−0.54, p=0.009 (FIG. 4F). To further evaluate whether the high variation in % Lp(a)-C was due to intra- or inter-individual differences, the mean (SD) intra-individual coefficient of variation (CV) of % Lp(a)-C across the 3 pre-determined study time points were determined to be 20.4 (10.2) % in the entire cohort. The CV of % Lp(a) was 21.6 (10.9) % in the IONIS-APO (a) Rx ASO group, 16.8 (7.5) % in the placebo group, without a statistically significant difference between the two treatment groups (p=0.23). Moreover, there was no statistically significant correlation between % Lp(a)-C and Lp(a) molar concentration (r=−0.04, p=0.7) nor Lp(a) mass (r=−0.2, p=0.1).

Direct LDL-C assays detect Lp(a)-C. To ascertain whether commercially-available direct LDL assays also detect Lp(a)-C, cholesterol was quantified on purified Lp(a) that was free of any LDL using a total cholesterol assay as gold standard in addition to 3 independent direct LDL-C assays. The cholesterol content of Lp(a) was 54.9 (1.3) [mean (SD)]mg/dL by the total cholesterol assay, 44.4 (0.6) mg/dl by the Roche LDLC3 assay, 57.3 (1.9) mg/dl by the Sekisui direct LDL-C assay, and 49.7 (2.3) mg/dL by the Wako L-Type LDL-C assay (Table 3). Using the total cholesterol on purified Lp(a) as a reference 87 (3.1) 8, 104 (5.4) 8, and 90 (6.2) % of Lp(a)-C was measured as LDL-C by the Roche, Sekiusi, and Wako assays, respectively. When purified Lp(a), with a total cholesterol content of 54.9 mg/dl, was spiked-into plasma from a patient with a total cholesterol of 155.6 mg/dl and Lp(a) mass of 5 mg/dl, 84 (2.3) %, 98 (0.2) ¿, and 98 (0.2) % of the additional cholesterol from the exogenously added Lp(a) was measured as LDL-C by the Roche, Sekiusi, and Wako assays, respectively (Table 3).

TABLE 3
Performance of 3 direct LDL-C assays with detection of cholesterol
on purified Lp(a) and Lp(a) spiked-in plasma.

| | Input | | | | |
|---|---|---|---|---|---|
| | | | Plasma + | % pure | % spiked-in |
| | Plasma | Purified | purified | Lp(a)-C | Lp(a)-C |
| | sample | Lp(a) | Lp(a) | detected | detected |
| Total cholesterol | 155.6 (3.2) | 54.9 (1.3) | 200.5 (0.6) | 100% | 100% |
| Roche direct LDL-C | 97.7 (2.2) | 44.4 (0.6) | 144.0 (1.1) | 87 (3.1)% | 84 (2.3)% |
| Sekiusi direct | 113.1 (6.4) | 57.3 (1.9) | 166.8 (3.3) | 104 (5.4)% | 98 (0.2)% |
| LDL-C | | | | | |
| Wako direct LDL-C | 105.1 (5.2) | 49.7 (2.3) | 158.9 (2.9) | 90 (6.2)% | 98 (0.1)% |

Purified Lp(a) with a cholesterol content of 54.9 mg/dL was used for these experiments. % pure Lp(a)-C detected = Lp(a)-C measured by the respective direct LDL-C assay divided by Lp(a)-C measured by the total cholesterol assay. % spiked-in Lp(a)-C = [cholesterol measured in plasma spiked with pure Lp(a) minus cholesterol measured in plasma without added Lp(a)] divided by cholesterol measured in purified Lp(a), using each respective assay. Data described are from 3 separate experiments and expressed as mean (SD). Units are in mg/dL unless otherwise specified.

### Example 2

Study population. A randomized, double-blind, placebo-controlled, dose-ranging trial involving 286 patients with established CVD and screening Lp(a) levels >60 mg/dL (>150 nmol/l). Patients received an Apo (a) lowering drug at 20, 40, or 60 mg every 4 weeks; 20 mg every 2 weeks; or 20 mg every week) (cumulative doses of 20, 40, 60 and 80 mg monthly), or saline placebo subcutaneously for 6 to 12 months. The primary end point was the percent change in Lp(a) levels from baseline to month 6 of exposure (week 25 in the groups that received monthly doses and week 27 in the groups that received more frequent doses).

Measurement of Lp(a)-cholesterol. The methodology and early clinical experience of the method for directly measuring Lp(a)-C is described herein (see, e.g., FIG. 1) and comprises adding plasma to monoclonal antibody LPA4 conjugated magnetic beads (MyOne Epoxy, Life Technologies) in the presence of 200 mM proline and 200 mM epsilon amino caproic acid (EACA) in U-bottom 96-well plates. The Lp(a)-LPA4-dynabeads are then extracted from each well using a magnetic bead extraction replicator and released into parallel 96-well plates containing 200 ul of PBS 18 BSA, 200 mM proline and 200 mM EACA in each well and any non-specifically bound cholesterol is wash off. The Lp(a)-LPA4-dynabeads are then transferred to a parallel, clear, flat-bottom, 96-well plate containing 200 ul of enzymatic cholesterol reagent (Pointe Scientific) and analyzed for absorbance at 500 nm (primary) and 700 nm (background). The amount of Lp(a)-C is determined against a standard curve, adjusting for the input volume of plasma. The 2 major advances of this assay compared prior methods is the high sensitivity to <1 mg/dL cholesterol and the expanded linear range of up 747 nmol/L Lp(a) that captures >99% of the population Lp(a) levels. In prior studies with electrophoretic methods, Lp(a)-C could only be quantitated in ˜⅓ of subjects whose levels were generally >30 mg/dL. In the current study, Lp(a)-C was measured at baseline, week 13, the primary analysis timepoint (week 25/27) and week 69/final analysis timepoint, which represents 16 weeks off the apo (a) lowering drug in all subjects.

Laboratory measurements. Relevant laboratory variables were measured. In brief, Lp(a) molar concentrations (nmol/L), representing apolipoprotein (a) particle number, were measured with an isoform-independent assay at the Northwest Lipid Metabolism and Diabetes Research Laboratories, University of Washington. All other laboratory measurements including LDL-C and total apoB were measured with commercially available kits at Medpace Reference Laboratories. Laboratory-reported LDL-C was calculated by the Friedewald formula or measured by ultracentrifugation if triglyceride levels exceeded 400 mg/dL. Laboratory measured LDL-C values were further corrected by 2 methods: 1—by the current method as LDL-CcorrDirectLp(a)=laboratory LDL-C-directly measured Lp(a)-C; and 2—by the Dahlen formula as LDL-CcorrDahlén=laboratory LDL-C-(Lp(a) mass * 0.30).

Levels of apoB not associated with Lp(a) (non-Lp(a)-apoB) were calculated by converting total apoB mass in mg/dL to nmol/L by multiplying by 19.9493 (using molecular weight of apoB at 550 kDa) and then subtracting Lp(a) molar concentration (Non Lp(a)-apoB=total ApoB-Lp(a)-apoB). Since apoB and apo (a) are in a 1:1 molar relationship, when the molar concentration of Lp(a) is known, the Lp(a)-apoB concentration is identical. Oxidized phospholipids on apoB (OxPL-apoB) and apo (a) (OxPL-apo (a) were measured.

Statistical analysis. All summaries and analyses were conducted in the Full Analysis set, defined as all patients who had undergone randomization and had received at least one dose of study drug or placebo. The baseline data were summarized using descriptive statistics. Continuous data are expressed as means and standard deviations or medians and IQRs. Correlations for the Day 1 pre-dose data were assessed by the Spearman's rank correlation coefficient. Correlations for the data at Primary Analysis Timepoint were assessed by the Spearman's partial rank order correlation coefficients controlling for treatment group and log-transformed baseline values. The percent changes in Lp(a)-C, Lp(a), laboratory-reported LDL-C, LDL-CcorrDirectLp(a), LDL-CcorrDahlén, total apoB, and non-Lp(a) apoB levels were analyzed with the use of an analysis of covariance model with treatment group as factor and the log-transformed baseline value for each respective measure as covariate. Missing data were handled with a multiple-imputation model containing baseline and postbaseline values, stratified according to treatment group. The imputations were performed for post baseline values by the Markov chain Monte Carlo method. Due to the exploratory nature of this analysis, the P values and widths of the 95% confidence intervals were not adjusted for multiplicity. The analyses were performed with SAS version 9.4.

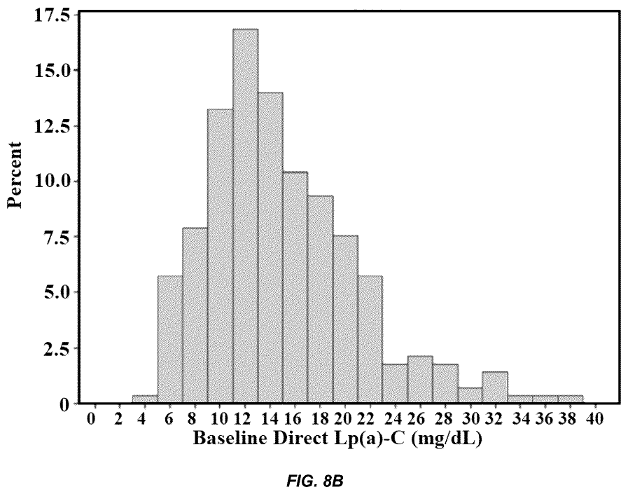

Baseline levels of laboratory parameters in the treatment groups. Table 4 displays the relevant laboratory variables of the current analysis. The baseline Lp(a) levels ranged from ˜205-247 nmol/L and the mean Lp(a)-C values ranged from 13.3-16.9 mg/dL. The laboratory reported LDL-C ranged from 69.5-89.5 in the 6 groups. The LDL-CcorrDirectLp(a) was ˜13-16 mg/dL lower than the laboratory reported LDL-C. In contrast, the LDL-CcorrDahlen was ˜27-33 mg/dL lower than the laboratory measured LDL-C, consistent with LDL-CcorrDahlen significantly overestimating Lp(a)-C and underestimating true LDL-C. The frequency distribution of baseline levels of Lp(a)-C(FIG. 8A) are right skewed and similar to Lp(a) molar concentration (FIG. 8B).

TABLE 4
Laboratory Characteristics of the Patients at Baseline

| | Drug therapy | | | | | | | |
|---|---|---|---|---|---|---|---|---|
| | 20 mg/Q4W | | 40 mg/Q4W | | 60 mg/Q4W | | 20 mg/Q2W | |
| | N = 48 | | N = 48 | | N = 47 | | N = 48 | |
| Lp(a), median (IQR), | 246.6 | (179.2, 300.8) | 220.0 | (176.5, 283.3) | 204.5 | (163.8, 286.5) | 238.2 | (183.7, 298.4) |
| nmol/L | | | | | | | | |
| Direct Lp(a)-C, median | 15.0 | (11.8-19.6) | 12.3 | (9.4-17.0) | 11.9 | (10.0-14.8) | 14.1 | (10.2-18.0) |
| (IQR), mg/dL | | | | | | | | |
| Lp(a)-C_{Dahlén}, median | 29.6 | (21.5-36.1) | 26.4 | (21.2-34.0) | 24.5 | (19.7-34.4) | 28.6 | (22.0-35.8) |
| (IQR), mg/dL | | | | | | | | |
| Laboratory-reported LDL-C, | 89.5 | (37.1) | 77.5 | (39.5) | 68.5 | (27.2) | 74.5 | (28.8) |
| mean (SD), mg/dL | | | | | | | | |
| LDL-C_{corr Direct Lp(a)}, mean | 73.5 | (33.9) | 63.3 | (37.1) | 55.2 | (26.9) | 59.2 | (27.1) |
| (SD), mg/dL | | | | | | | | |
| LDL-C_{corr Dahlén}, mean(SD), | 56.5 | (39.5) | 49.9 | (39.2) | 40.9 | (28.3) | 45.3 | (27.7) |
| mg/dL | | | | | | | | |
| Total Apo B, mean (SD), | 80.7 | (23.6) | 71.9 | (23.4) | 68.5 | (18.8) | 69.3 | (19.8) |
| mg/dL | | | | | | | | |
| Total Apo B, mean (SD), | 1466.7 | (429.4) | 1306.4 | (424.6) | 1245.3 | (341.4) | 1259.8 | (359.5) |
| nM | | | | | | | | |
| Non-Lp(a) apoB, mean (SD), | 1204.1 | (426.7) | 1066.0 | (426.2) | 1012.6 | (355.8) | 1004.9 | (356.9) |
| nM | | | | | | | | |
| Total cholesterol, mean (SD), | 166.0 | (38.6) | 154.4 | (52.1) | 142.9 | (32.4) | 146.7 | (36.3) |
| mg/dL | | | | | | | | |
| Triglycerides, median (IQR), | 97 | (44-230) | 97 | (35-283) | 106 | (53-567) | 106 | (35-204) |
| mg/dL | | | | | | | | |
| HDL-C, mean (SD), mg/dL | 54.2 | (15.9) | 55.7 | (21.0) | 48.9 | (12.8) | 52.8 | (18.8) |

| | Drug therapy | | Pooled | |
|---|---|---|---|---|
| | 20 mg/QW | | placebo | |
| | N = 48 | | N = 47 | |
| Lp(a), median (IQR), | 233.7 | (193.1, 275.3) | 231.6 | (194.9, 317.7) |
| nmol/L | | | | |
| Direct Lp(a)-C, median | 14.3 | (11.7-18.5) | 15.6 | (11.5-20.2) |
| (IQR), mg/dL | | | | |
| Lp(a)-C_{Dahlén}, median | 28.0 | (23.2-33.0) | 27.8 | (23.4-38.1) |
| (IQR), mg/dL | | | | |
| Laboratory-reported LDL-C, | 76.2 | (28.5) | 78.7 | (29.8) |
| mean (SD), mg/dL | | | | |
| LDL-C_{corr Direct Lp(a)}, mean | 61.5 | (27.9) | 62.5 | (29.0) |
| (SD), mg/dL | | | | |
| LDL-C_{corr Dahlén}, mean(SD), | 46.9 | (30.1) | 47.7 | (30.1) |
| mg/dL | | | | |
| Total Apo B, mean (SD), | 70.6 | (19.1) | 73.8 | (16.9) |
| mg/dL | | | | |
| Total Apo B, mean (SD), | 1283.7 | (348.2) | 1342.4 | (307.7) |
| nM | | | | |
| Non-Lp(a) apoB, mean (SD), | 1043.6 | (367.3) | 1091.6 | (306.2) |
| nM | | | | |
| Total cholesterol, mean (SD), | 154.4 | (34.7) | 154.4 | (33.6) |
| mg/dL | | | | |
| Triglycerides, median (IQR), | 89 | (35-266) | 106 | (35-576) |
| mg/dL | | | | |
| HDL-C, mean (SD), mg/dL | 57.6 | (18.4) | 51.6 | (18.2) |

Data are mean (SD) unless otherwise noted. Lp(a) in mg/dL was calculated by dividing values in molar concentration by 2.5. To convert the values for cholesterol to millimoles per liter, multiply by 0.02586. To convert the values for triglycerides to millimoles per liter, multiply by 0.01129. QW, once a week; Q2W, every 2 weeks; Q4W, every 4 weeks; SD, standard deviation; TIA, transient ischemic attack.

The effect of apo (a) lowering drug on Lp(a) and Lp(a)-C levels. Treatment drug resulted in statistically significant dose-dependent mean percent decreases in Lp(a)-C compared with 2% decrease in pooled placebo (29%-678, P-values 0.001-<0.0001 at the primary analysis timepoint). The extent and temporal changes in directly measured Lp(a)-C are consistent with change in Lp(a) molar concentration. Spearman p between Lp(a)-C and Lp(a) molar concentration at baseline and the primary analysis timepoint (week 25/27) were 0.72 (p<0.0001) and 0.58 (P<0.0001), respectively.

Compared to the laboratory-derived LDL-C, the LDL-CcorrDirectLp(a) trended in the same direction, albeit with a smaller effect and with loss of statistical significance in some of the groups. In contrast, the estimated levels of LDL-CcorrDahlén were significantly increased from baseline in most of the cohorts compared to placebo. Reflecting the limitations of the Dahlén formula, there were 8 patients who had only baseline LDL-CcorrDahlén<=0, 2 patients who had LDL-CcorrDahlen at the PAT <=0, and 4 patients who had LDL-CcorrDahlen at both baseline and PAT <=0. Total apoB levels were significantly decreased in all cohorts except the lowest dose at 20 mg/4 wk. However, the non Lp(a)-apoB levels were not significantly reduced in any cohorts.

Correlations between Lp(a)-related variables. In all groups combined at baseline, direct Lp(a)-C strongly correlated with Lp(a) molar concentration (p<0.0001), modestly with laboratory-reported LDL-C, total apoB, OxPL-apoB and triglycerides (P<0.0001 for all) and weakly with LDL-CcorrDirectLp(a) (p=0.0077). LDL-CcorrDirectLp(a) did not correlate with Lp(a) or direct Lp(a)-C but did correlate strongly with total apoB and non-Lp(a) apoB. In all groups combined at the primary analysis timepoint, generally similar trends in statistical associations were noted.

The study demonstrates several findings. First, it demonstrates that the test drug produced a robust, dose-dependent reduction in directly measured Lp(a)-C that is consistent with its effect on Lp(a) molar concentration. Second, it demonstrates the utility of using directly measuring Lp(a)-C to derive a more accurate LDL-C, showing corrected LDL-C is 13-16 mg/dL lower than laboratory-reported LDL-C. Third, it confirms that the Dahlén formula, which uses a fixed 30% correction of Lp(a) mass to estimate Lp(a)-C for every individual to derive a corrected LDL-C, is erroneous and should be discontinued. The clinical implications of each finding are discussed below.

The dose-dependent reduction in Lp(a)-C is a novel observation and mirrored the reduction in Lp(a) molar concentration but was of slightly lower magnitude. The reduction in Lp(a)-C is not unexpected since all patients had elevated Lp(a) levels and the test drug has been shown to potently lower Lp(a) with no non-responders identified to date. The correlations between the baseline and primary endpoint of Lp(a)-C and Lp(a) molar concentration were strong and statistically significant but not near unity. This confirms that Lp(a) particles in subjects with highly elevated Lp(a) are heterogeneous in their cholesterol content, ranging from 6-57% in an inverse and curvilinearly fashion as recently shown.

The data showed that baseline LDL-CcorrDirectLp(a) levels were ˜13-16 mg/dL lower than the laboratory-reported LDL-C, a clinically significant difference based on the CTT meta-analysis. Additionally, in a meta-analysis of landmark statin trials including 18,043 patients, 5390 events and 4.7 years median follow-up that mathematically removing Lp(a)-C from the laboratory reported LDL-C in a bracket of 20-45% of Lp(a) mass resulted in corrected LDL-C no longer being predictive of MACE. The current study is an advance over this prior work in now providing a quantitative method to assess the LDL-C more accurately in patients with elevated Lp(a). With the emergence of Lp(a) lowering therapeutics, the ability to differentiate a more accurate LDL-C from Lp(a)-C may allow the ability to assess which pool of cholesterol will be responsive to LDL-targeted versus Lp(a)-targeted therapies and to choose the most appropriate types and dosage of concomitant therapies.

The trends in changes in LDL-CcorrDirectLp(a) are largely reflected in the laboratory LDL-C, but with an attenuation of the effect size leading to lack of statistical significance in all but one treatment group. This suggests that the laboratory-reported LDL-C mildly overestimates the LDL-C lowering effect, likely because it includes the Lp(a)-C that was reduced in significantly greater proportion than true LDL-C. The current technique most closely resembles the most accurate LDL-C changes due to drug therapy and that both laboratory-reported LDL-C and the Dahlén formula are less accurate.

It was also demonstrated here that the Dahlén formula, by assuming that Lp(a)-C is universally a fixed 30% of Lp(a) mass, overestimates Lp(a)-C and underestimates true LDL-C in patients with elevated Lp(a), where it is most crucial to differentiate the proportions of each. The original description of this formula was reported in textbook format and appears to have been studied in a small number of subjects without subsequent rigorous biochemical or clinical validation. While Lp(a)-C measured using an indirect method in 55 Japanese individuals suggested an average Lp(a)-C mg/dL to Lp(a) mg/dL ratio of 0.3, significant inter-individual variation existed. Lp(a) mass assays (mg/dL) measure apo (a) immunologically and not total particle mass and use calibrators not traceable to any validated primary standard, thus the denominator of this equation is a source of error. When an Lp(a)-C mass is needed, a directly measured Lp(a)-C should be used to allow determination of Lp(a)-C and LDL-C.

The observation that the significant reduction in total apoB levels appeared to be driven by reduction in Lp(a)-apoB implies enhanced apoB plasma clearance. It is known that clearance of Lp(a) particles is slower than LDL particles (29-31), likely due to weaker recognition of Lp(a)-apoB by LDL receptors.

The current method to quantitate Lp(a)-C is useful in understanding changes in lipid components affecting both Lp(a)-C and LDL-C.

A number of embodiments have been described herein. Nevertheless, it will be understood that various modifications may be made without departing from the spirit and scope of this disclosure. Accordingly, other embodiments are within the scope of the following claims.

1. A method of assaying lipoprotein (a)-cholesterol (Lp(a)-C) in a sample, the method comprising:
contacting a sample with a binding molecule that specifically binds to apolipoprotein (a) (apo (a)) to obtain an apo (a)-binding molecule complexes;
isolating the apo (a)-binding molecule complexes;
performing an enzymatic colorimetric assay on the isolated apo (a)-binding molecule complexes to measure cholesterol.

2. The method of claim 1, wherein the method further comprises removing the apo (a)-binding molecule complexes and measuring the absorbance of the sample.

3. The method of claim 1, wherein the binding molecule comprises light chain and heavy chain complement determining regions (CDRs) of an LPA4 antibody.

4. The method of claim 1, wherein the binding molecule is an antibody or antibody fragment.

5. The method of claim 4, wherein the antibody or antibody fragment recognizes and binds to lipoprotein (a), wherein the antibody or antibody fragment comprises a variable heavy chain (V_{H}) domain and/or a variable light chain (V_{L}) domain, and wherein (a) the V_{H} domain comprises an amino acid sequence that includes complementarity determining regions (CDRs) selected from the group consisting of: SEQ ID NO:4 or variants thereof; SEQ ID NO:6 or variants thereof; and SEQ ID NO:8 or variants thereof; and (b) the V_{L} domain comprises an amino acid sequence that includes complementarity determining regions (CDRs) selected from the group consisting of: SEQ ID NO: 12 or variants thereof; SEQ ID NO:14 or variants thereof; and SEQ ID NO: 16 or variants thereof.

6. The method of claim 5, wherein the V_{H} domain comprises an amino acid sequence of SEQ ID NO:2, and/or the V_{L} domain comprises an amino acid sequence of SEQ ID NO:10.

7. The method of claim 4, wherein the antibody or antibody fragment is selected from the group consisting of an antibody and scFv with heavy and light chain domains comprising the complementarity determining regions of SEQ ID NO:4, 6, 8, 12, 14, and 16.

8. The method of claim 4, wherein the antibody or antibody fragment binds to an epitope having the sequence of SEQ ID NO: 17.

9. The method of claim 1, wherein the binding molecule is linked to a substrate.

10. The method of claim 9, wherein the substrate is an ELISA plate or a bead.

11. (canceled)

12. The method of claim 10, wherein the bead is a magnetic bead.

13. The method of claim 1, wherein the sample is from a subject undergoing therapy for high cholesterol.

14. A method of assaying lipoprotein (a)-cholesterol (Lp(a)-C) in a sample, the method comprising:
contacting a sample with an antibody or antibody fragment that specifically binds to apolipoprotein (a) (Apo (a)) to obtain and Apo (a)-antibody complex;
isolating the apo (a)-antibody complexes; and
measuring the amount of cholesterol in the isolated Apo (a)-antibody complex.

15. The method of claim 14, wherein the antibody is bound to a substrate.

16. The method of claim 15, further comprising washing the substrate to remove non-bound materials.

17. The method of claim 14, wherein the antibody is bound to a bead.

18. The method of claim 17, wherein the bead is magnetic.

19. The method of claim 18, wherein the Apo (a)-antibody complex is isolated using a magnet.

20. The method of claim 1, wherein cholesterol is measured using a colorimetric enzymatic assay.

21. (canceled)

22. The method of claim 14, wherein the method determines the level of cholesterol in an Apo (a) containing fraction of plasma.

23. The method of claim 14, wherein the antibody or antibody fragment comprises a variable heavy chain (V_{H}) domain and/or a variable light chain (V_{L}) domain, and wherein (a) the V_{H} domain comprises an amino acid sequence that includes complementarity determining regions (CDRs) of SEQ ID NO:4 or variants thereof; SEQ ID NO:6 or variants thereof; and SEQ ID NO:8 or variants thereof; and (b) the V_{L} domain comprises an amino acid sequence that includes complementarity determining regions (CDRs) of SEQ ID NO:12 or variants thereof; SEQ ID NO:14 or variants thereof; and SEQ ID NO: 16 or variants thereof.

24. The method of claim 14, wherein the antibody or antibody fragment is selected from the group consisting of an antibody and scFv with heavy and light chain domains comprising the complementarity determining regions of SEQ ID NO:4, 6, 8, 12, 14, and 16.

25. A method of assaying low density lipoprotein cholesterol (LDL-C) in a sample, the method comprising:
contacting a plasma sample with a composition or article of manufacture comprising a binding agent linked to a carrier, wherein the binding agent specifically binds to Lp(a) and wherein the carrier separates bound Lp(a) (Lp(a)-C fraction) from a soluble fraction of the plasma and measuring the amount of cholesterol in the soluble fraction of the plasma thereby obtaining LDL-C value.

26. The method of claim 25, wherein the binding agent is an antibody that specifically binds to Lp(a).

27. The method of claim 25, wherein the antibody is a polyclonal antibody or monoclonal antibody.

28-29. (canceled)

30. The method of claim 27, wherein the antibody comprises one or more CDRs having sequences of SEQ ID NO: 4, 6, 8, 12, 14, and/or 16.

31. The method of claim 25, further comprising measuring the amount of cholesterol in the Lp(a)-C fraction to obtain a Lp(a)-C value.

32. The method of claim 25, further comprising measuring the amount of total cholesterol in the sample or a corresponding sample prior to contacting the sample, with the composition or article of manufacture to obtain an LDL-C value.

33. (canceled)

34. The method of claim 32, wherein the Lp(a)-C value is subtracted from the total cholesterol value to obtain a corrected LDL-C value.

35. The method of claim 25, wherein the composition comprises a bead linked to the binding agent.

36. The method of claim 35, wherein the bead is a magnetic bead.

37. (canceled)

38. The method of claim 25, wherein the article of manufacture is a substrate comprising the binding agent.

39. The method of claim 38, wherein the substrate is a microwell plate.

40. (canceled)
